(19) United States
(12) Patent Application Publication
(10) Pub. No.: US 2001/0021514 A1
(43) Pub. Date: Sep. 13, 2001

(54) Luminescent metal-ligand complexes

(75) Inventors: Ewald A. Terpetschnig, Sunnyvale, CA (US); Dan-Hui Yang, Sunnyvale, CA (US); John C. Owicki, Palo Alto, CA (US); Yan Zhang, Los Altos, CA (US)

Correspondence Address: KOLISCH, HARTWELL, DICKINSON,, McCORMACK & HEUSER, Suite 200, 520 S.W. Yamhill Street, Portland, OR, 97204, US

(73) Assignee: LJL BioSystems, Inc.

(21) Appl. No.: 09/767,583

(22) Filed: Jan. 22, 2001

Related U.S. Application Data
(63) Continuation of application No. PCT/US00/03589, filed on Feb. 11, 2000.
(60) Provisional application No. 60/119,884, filed on Feb. 12, 1999.
(60) Provisional application No. 60/165,813, filed on Nov. 16, 1999.

(51) Int. Cl. C09K 11/06; C12Q 1/68; G01N 33/53; G01N 33/537; G01N 33/543

(52) U.S. Cl. 435/7.92; 252/301.16; 435/6

(57) ABSTRACT

Luminescent metal-ligand complexes and/or complementary energy transfer acceptors for use in luminescence assays. The complexes and/or acceptors may be used in free, reactive, and/or conjugated form, alone or mixed with other compounds. Preferred luminescence assays include luminescence polarization and luminescence resonance energy transfer assays, among others.

### BACKGROUND OF THE INVENTION

A luminescent compound, or luminophore, is a compound that emits light. A luminescence assay, in turn, is an assay that involves detecting light emitted by a luminophore and using properties of that light to understand properties of the luminophore and its environment. Luminescence assays may be based on photoluminescence and chemiluminescence, among others. Luminescence assays may include immunoassays, binding/hybridization assays, and cleavage/digestion assays, among others, and competition assays and sandwich assays, among others.

Luminescence assays may have significant advantages over nonluminescence-based assays, such as radioassays. First, luminescence assays may be very sensitive, because modern detectors, such as photomultiplier tubes (PMTs) and charge-coupled devices (CCDs), can detect very low levels of light. Second, luminescence assays may be very selective, because the luminescence signal may come almost exclusively from the luminophore.

Despite these potential strengths, luminescence assays suffer from a number of shortcomings, at least some of which relate to the luminophore. The luminophore may have an extinction coefficient and/or quantum yield that is too low to permit detection of an adequate amount of light. The luminophore also may have a Stokes' shift that is too small to permit detection of emission light without significant detection of excitation light. The luminophore also may have an excitation spectrum that does not permit it to be excited by wavelength-limited light sources, such as lasers and arc lamps; for example, the argon-ion laser generates significant light only at wavelengths of about 488 and 514 nm. The luminophore also may be unstable, so that it is readily bleached and rendered nonluminescent. The luminophore also may have excitation and/or emission spectra that overlap with the autoluminescence of biological and other samples; such autoluminescence is particularly significant at wavelengths below about 600 nm. The luminophore also may be expensive, especially if it is difficult to manufacture.

Luminescence assays directed to particular purposes or involving measurement of particular quantities may be subject to additional limitations. For example, luminescence polarization assays, which are used to monitor molecular reorientation, typically involve matching a luminescence lifetime to a rotational correlation time. Yet, the lifetime of many luminophores is too short for monitoring rotation of many analytes. Generally, rotational correlation times increase by about 1 nanosecond for each 2,400 Daltons in molecular weight. Most luminophores used in polarization assays have luminescence lifetimes near 4 nanoseconds; such luminophores only may be used to monitor rotation of analytes with molecular weights less than several thousand Daltons. Moreover, polarization assays also typically employ probes with high intrinsic polarizations. Yet, the intrinsic polarization of many luminophores is too low to monitor rotation. Generally, the polarization varies between about zero and about the intrinsic polarization in a polarization experiment. Thus, if the intrinsic polarization is low (i.e., near zero), the assay will not have enough range to monitor rotation.

Similarly, luminescence energy transfer assays, which may be used to monitor molecular proximity, typically involve matching an energy transfer donor and an energy transfer acceptor. Yet, the numbers of such compounds is limited. In addition, the lifetimes of known donors and acceptors typically are short, so that lifetime signals from these molecules may be measurable only with high-frequency detectors and may be confused with lifetime signals from background luminophores. Moreover, the excitation and emission wavelengths of known donors and acceptors may be in the ultraviolet or infrared, potentially requiring exotic filter sets and exposing operators to dangerous radiation. Further, Stokes shifts for known donors and acceptor pairs may be small, making it difficult to separate donor and acceptor luminescence.

### SUMMARY OF THE INVENTION

The invention provides luminescent metal-ligand complexes and/or complementary energy transfer acceptors for use in luminescence assays. The complexes and/or acceptors may be used in free, reactive, and/or conjugated form, alone or mixed with other compounds. Preferred luminescence assays include luminescence polarization and luminescence resonance energy transfer assays, among others.

### BRIEF DESCRIPTION OF THE DRAWINGS

FIG. 1 shows examples of luminescent metal-ligand complexes with exclusively chromophoric ligands for use in luminescence assays.

FIG. 2 shows examples of luminescent metal-ligand complexes with both chromophoric and nonchromophoric ligands for use in luminescence assays.

FIG. 3 shows examples of energy transfer pairs comprising a luminescent metal-ligand-complex donor and an acceptor capable of accepting energy transfer from the donor for use in energy transfer assays.

FIG. 4 is a schematic view of luminescently labeled molecules, showing how molecular reorientation affects luminescence polarization.

FIG. 5 is a graph showing the relationship between rotational correlation time τᵣₒₜ and luminescence lifetime τ for use in polarization assays.

FIG. 6 is a graph showing the steady-state luminescence polarization of Sunnyvale Red™-HSA at various concentrations of anti-HSA antibody (full line) or nonspecific antibody (IgG) (dashed line).

FIG. 7 is a graph showing the steady-state luminescence polarization of fluorescein-HSA at various concentrations of anti-HSA antibody (full line) or nonspecific antibody (IgG) (dotted line).

FIG. 8 is a graph showing absorption and emission spectra of a Ru(bpy)(phen-ITC)²⁺ donor and a LGY-HSA acceptor.

FIG. 9 is a graph showing relative intensities for the titration of ruthenium-labeled antibody with LGY-labeled human serum albumin.

FIG. 10 is a graph showing phase and modulation frequency responses for the titration of a ruthenium-labeled antibody with acceptor-labeled human serum albumin.

FIG. 11 is a flowchart showing a synthetic scheme for Ru(4-aminomethyl-4′-methyl-2,2′-bipyridine)₂(dcbpy) and Ru(4-aminomethyl-4′-methyl-2,2′-bipyridine)₂ (dmcbpy).

FIG. 12 is a flowchart showing a synthetic scheme for Ru(Phen-NH₂)₂(dcbpy) and Ru(phen-NH₂)₂(dmcbpy) isothiocyanate.

FIG. 13 is a flowchart showing a synthetic scheme for Sunnyvale Red™ and its mono-reactive version.

FIG. 14 is a flowchart showing a synthetic scheme for the aromatic version of Sunnyvale Red™.

FIG. 15 is a flowchart showing a synthetic scheme for Fair Oaks Red™ and Ru(Phen-NH₂)₃ ITC.

FIG. 16 is a flowchart showing a synthetic scheme for reactive Ru-diphenylphenanthroline derivatives.

FIG. 17 is a flowchart showing a synthetic scheme for reactive Ru-diphenylbipyridine derivatives.

FIG. 18 is a flowchart showing a synthetic scheme for Ru-tris(bathophenantroline).

FIG. 19 is a flowchart showing a synthetic scheme for a mono-chromophoric Os-phosphino-complex.

FIG. 20 is a flowchart showing a synthetic scheme for a mono-chromophoric Ru-phosphino-complex.

FIG. 21 is a flowchart showing a synthetic scheme for Fast Green FCF-NHS ester.

Abbreviations

| Abbreviation | Term |
|---|---|
| bpy | 2,2′-bipyridine |
| dcbpy | 4,4′-dicarboxyl-2,2′-bipyridine |
| dcsubpy | 4,4′-dicarboxysuccinimidyl-2,2′-bipyridine |
| DMF | N,N-dimethylformamide |
| dppe | 1,2-Bis(diphenylphosphino)ethane |
| dppy | Cis-1,2-Bis(diphenylphosphino)ethylene |
| HSA | Human serum albumin |
| lgG | Immunoglobulin G, human |
| ITC | Isothiocyanate |
| mcbpy | 4-methyl-4′-carboxyl-2,2′-bipyridine |
| mcsubpy | 4-methyl-4′-carboxysuccinimidyl-2,2′-bipyridine |
| phen-NH₂ | 9-amino-1,10-phenanthroline |
| phen-ITC | 1,10-phenanthroline-9-isothiocyanate |
| tsNadppb | 1,2-Bis(di-4-sulfonatophenylphosphino)benzene |
| | tetrasodium salt |
| TSU | O-(N-succinimidyl)-N,N,N′,N′-tetramethyluronium |
| | tetrafluoroborate |

### DETAILED DESCRIPTION OF THE INVENTION

The invention relates to luminescent metal-ligand complexes, to energy transfer acceptors for use with such complexes, and to methods and kits for synthesizing and using such complexes and acceptors. The complexes and acceptors may be useful as probes, labels, and/or indicators in luminescence assays, in free, reactive, and/or conjugated form, alone or mixed with other compounds. This usefulness may reflect an enhancement of one or more of the following properties: intrinsic polarization (or, equivalently, fundamental anisotropy), quantum yield, luminescence lifetime, Stokes' shift, and extinction coefficient, among others. This usefulness also may reflect luminescence lifetimes that match rotational correlation times of analytes of interest. This usefulness also may reflect absorption and emission spectra that complement particular light sources or detectors, respectively, or that permit excitation and/or emission at wavelengths inadequately covered by existing compounds. These and other aspects of the invention are described below, as follows: (A) compositions, (B) assays, and (C) synthetic/labeling procedures.

### A. Compositions

Aspects of the invention include compositions comprising a luminescent metal-ligand complex and/or an energy transfer acceptor capable of receiving energy transfer from such a metal-ligand complex.

1. Metal-Ligand Complexes

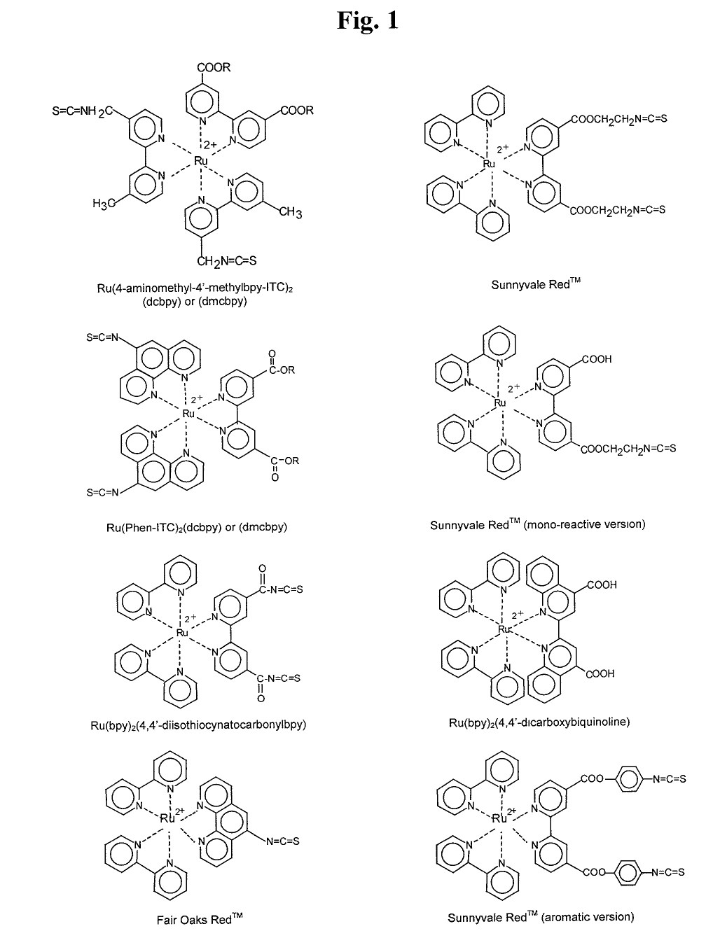

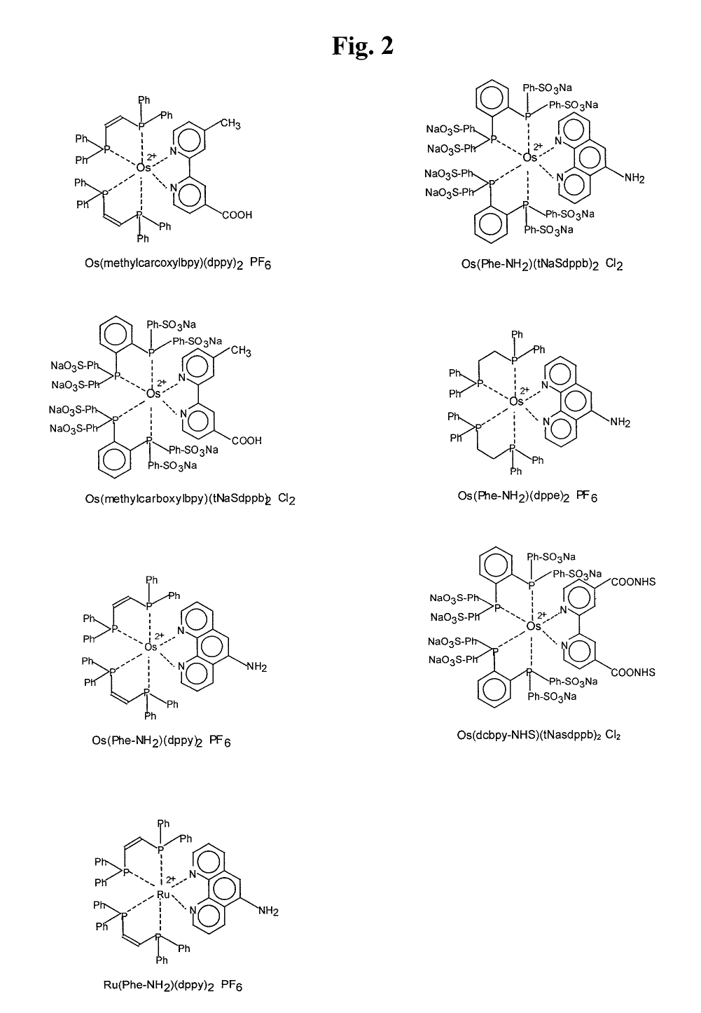

FIGS. 1 and 2 show luminescent metal-ligand complexes in accordance with aspects of the invention. A luminescent metal-ligand complex, as used herein, is a complex between a transition-metal (such as, without limitation, Ru(II), Re(I), or Os(II)) and one or more ligands, where the complex displays molecular photoluminescence arising from a metal-to-ligand charge-transfer state. These complexes generally have long luminescence lifetimes, where a long lifetime is defined as any lifetime greater than about 10 ns and preferably greater than about 50 or 100 ns. The ligands may include any molecule capable of coordinating with the metal. For example, the ligands may be chromophoric or nonchromophoric, and monodentate or polydentate, as described below.

“Chromophoric ligands” are colored due to selective light absorption, whereas “nonchromophoric ligands” are uncolored. Chromophoric ligands include aromatic pyridine compounds, whose metal-ligand charge-transfer states are relatively low in energy and so absorb visible light. Nonchromophoric ligands include carbon monoxide, halides, arsines, and phosphines, whose metal-ligand charge-transfer states are relatively high in energy.

“Monodentate ligands” are complexed to the metal at only one site on the ligand. Suitable monodentate ligands include carbon monoxide, cyanides, isocyanides, halides, and aliphatic, aromatic, and heterocyclic phosphines, amines, stibines, and arsines, among others.

“Polydentate ligands” are complexed to the metal at two or more sites on the ligand. Polydentate ligands include aromatic and aliphatic ligands, as well as aliphatic, aromatic, and heterocyclic phosphines, amines, stibines, and arsines, among others. Suitable aromatic polydentate ligands include aromatic heterocyclic ligands. Preferred aromatic heterocyclic ligands include nitrogen, such as bipyridyl, bipyrazyl, bipyrimidinyl, terpyridyl, and phenanthrolyl, among others.

The ligands may be unsubstituted, or substituted by any of a large number of substituents. Suitable substituents include alkyl, substituted alkyl, aryl, substituted aryl, aralkyl, substituted aralkyl, carboxylate, carboxaldehyde, carboxamide, cyano, amino, hydroxy, imino, hydroxycarbonyl, aminocarbonyl, amidine, guanidinium, ureide, sulfur-containing groups, phosphorus-containing groups, and various reactive groups, as described below, among others.

Generally, each ligand in a complex may independently be chromophoric or nonchromophoric, and polydentate or monodentate, subject to the limitation that the total number of ligand coordination sites in the complex equal the coordination number of the metal. However, usually at least one of the ligands in a complex is chromophoric, and at least one of the ligands is polydentate. FIG. 1 shows metal-ligand complexes in which each ligand is chromophoric, and FIG. 2 shows metal-ligand complexes in which at least one ligand is nonchromophoric.

The preferred properties of the metal-ligand complexes depend on the assay, such that the preferred properties differ between polarization and energy transfer assays, as described below. However, in most assays, it is preferable to have a high extinction coefficient, meaning that the complex has a high light-absorbing power.

A. Polarization Assays

As described below, polarization assays involve the absorption and subsequent re-emission of polarized light. In the polarization assays provided by the invention, the metal-ligand complex preferably will have a high intrinsic polarization, meaning that if the complex is immobilized it will emit substantially polarized light in response to excitation with polarized light. In this way, depolarization of light emitted from the complex will reflect molecular reorientation and/or environmental effects, rather than intrinsic properties of the complex. Symmetric molecules typically have low intrinsic polarization, and metal ions in solution typically have zero intrinsic polarization. In contrast, the invention provides compounds with enhanced intrinsic polarization, and methods for selecting and preparing such compounds. In particular, the intrinsic polarization can be increased by increasing the asymmetry of the complex. Asymmetry helps to define the absorption and emission dipoles involved in polarized excitation and emission, respectively.

Asymmetry can be created by combining chromophoric and nonchromophoric ligands, as shown in FIG. 2.

Asymmetry also can be created by additions and/or substitutions of electron-withdrawing and/or electron-donating groups to the ligands, as shown in FIG. 1 for complexes having exclusively chromophoric ligands. Electron-withdrawing and electron-donating groups alter the charge transfer distribution of the complex and hence the absorption and emission dipoles of the complex. Suitable electron-withdrawing groups include carboxyl, sulfoxyl, and amido groups, among others. Suitable electron-donating groups include alkyl, alkyloxy, and amino groups, among others.

In preferred complexes, at least one ligand includes a constituent of the form —C—(O)ₘ—Q₁—R₁, where m is 0 or 1, Q₁ is an alkyl or aryl group, R₁ is —N═C═S or —NH—C—NH—P, and P is a carrier (as described below). In some embodiments, the same or another ligand may include a second constituent, such as —C—(O)ᵤ—Q₂—(R₂)_{w}, where R₂ is a reactive group or a coupling to a P′, u is 0 or 1, w is 0 or 1, and Q₂ is an alkyl or aryl group. Here, P′ is a carrier that may be different than or the same as P. If P′ is different than P, then the metal ligand complex is a crosslinker, and if P′ is the same as P, then the metal ligand complex is a bifunctional reagent, attached to P at two sites. Bifunctional reagents may improve polarization properties in polarization experiments by reducing dye wobble (the “propeller effect”), so that the reagent reports on the motion of the carrier and not on its own independent motion.

The preferred complexes offer a number of advantages for polarization assays. The advantages include an electron-withdrawing carbonyl (i.e., —C═O) or carboxyl (i.e., —COO—) group, which provides asymmetry and hence enhanced polarization. The advantages also include an alkyl or aryl spacer group that positions the metal-ligand complex away from P, facilitating reaction. Preferred spacers comprise ethyl and phenyl groups, which provide adequate spacing without creating excessive flexibility that might reduce polarization. The advantages also include a reactive isothiocyanate group or an isothiourea linkage for attachment to an analyte or other molecule of interest. The isothiocyanate group is amine reactive, so that the complex can be attached to any carrier having (or modified to have) a free amino group. In some embodiments, the isothiocyanate group (or isothiourea bond) may be replaced by an isocyanate group (or isourea bond).

A preferred metal-ligand-complex polarization probe is Sunnyvale Red™. This complex has an excitation maximum at 488 nm and an emission maximum at 670 nm, corresponding to a Stokes' shift of nearly 200 nm. This complex also has a luminescence lifetime of about 360 ns and an intrinsic polarization P₀ of about 0.37 (corresponding to a fundamental anisotropy r₀ of about 0.28). This complex also has a minimum usable concentration of about 10 nM ligand/number of labels per protein in polarization assays using an Analyst™ light-detection platform (LJL BioSystems, Inc.).

B. Energy Transfer Assays

As described below, energy transfer assays involve the absorption of light by a luminescent energy transfer donor and the subsequent transfer of excited-state energy associated with this light to an energy transfer donor. In the energy transfer assays provided by the invention, the metal-ligand complex (such as that described above) is used as a donor. The complex preferably will have a high quantum yield, for example, at least about 4 percent, which generally is correlated with a long luminescence lifetime. In this way, the complex will be useful as an energy transfer donor. In particular, the rate of energy transfer from donor to acceptor is proportional to the quantum yield of the donor, so that a high quantum yield will increase the rate of transfer. Moreover, a high quantum yield will give the assay more dynamic range, because a greater fraction of the absorbed light can be diverted by energy transfer, further reducing the intensity of donor emission and the lifetime of the donor.

Preferred energy transfer metal-ligand-complex donors have long lifetimes, visible excitation, and large Stokes' shifts. A preferred donor is Fair Oaks Red™.

2. Energy Transfer Acceptors.

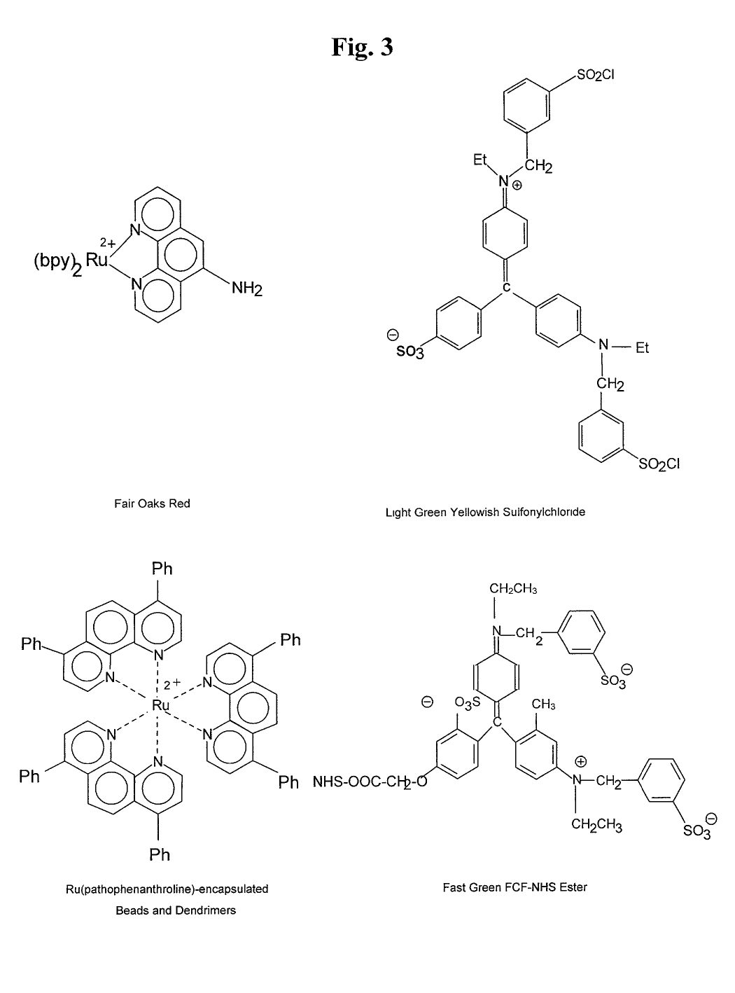

FIG. 3 shows examples of energy transfer acceptors and pairs of energy transfer donors and acceptors in accordance with the invention. The energy transfer acceptors are selected for their ability to accept excited-state energy from a metal-ligand-complex donor. In particular, this requires that the acceptor absorption spectrum overlap with the donor emission spectrum. Suitable acceptors include Light Green Yellowish sulfonylchlorid (LGY) and Isosulfan Blue. Acceptors may be luminescent or nonluminescent (dark), and may be bound covalently or noncovalently to the analyte or other molecule of interest.

3. Conjugates/Mixtures

In other aspects of the invention, these metal-ligand complexes and acceptors may be used free or conjugated to and/or mixed with other compounds. For example, the invention includes conjugates of the complexes and acceptors (or combinations thereof) with carriers, as described below. The invention also includes mixtures of the complexes and acceptors with themselves and/or with other luminophores and chemical moieties. Components of mixtures that include multiple luminophores may be distinguishable based on differences in their spectra and/or differences in their luminescence lifetimes.

4. Definitions

“Alkyl” denotes a branched or unbranched, saturated or unsaturated hydrocarbon radical. Suitable alkyl radicals include structures containing one or more methylene, methine, and/or methyne groups, among others. Branched structures may have a branching motif similar to i-propyl, t-butyl, i-butyl, and 2-ethylpropyl, among others. As used here, alkyl also includes substituted alkyls.

“Aryl” denotes an aromatic substituent, which may be a single aromatic ring or multiple aromatic rings that are fused together, linked covalently, or linked to a common group such as a methylene or ethylene moiety. The common linking group also may be a carbonyl, as in benzophenone. The aromatic ring(s) may include phenyl, napthyl, biphenyl, diphenylmethyl, and benzophenone, among others. Aryl also includes arylalkyl. As used here, aryl also includes substituted aryls.

“Carrier” denotes any molecule or other substrate capable of binding to a metal-ligand complex and/or acceptor as provided by the invention. Suitable carriers include biological substances, beads, polymers, and solid supports, among others. Biological substances may include whole cells, viruses, subcellular particles, proteins, lipoproteins, glycoproteins, polypeptides, nucleic acids, polysaccharides, lipopolysaccharides, cellular metabolites, hormones, pharmacological agents, tranquilizers, barbiturates, alkaloids, steroids, vitamins, amino acids, and sugars, among others. Whole cells may be animal, plant, fungal, or bacterial, among others, and may be alive or dead. Subcellular particles may include subcellular organelles, membrane particles as from disrupted cells, fragments of cell walls, ribosomes, multi-enzyme complexes, and other particles that can be derived from living organisms, among others. Nucleic acids may include chromosomal DNA, plasmid DNA, viral DNA, and recombinant DNA derived from multiple sources, among others. Nucleic acids also may include RNA, including messenger RNA, ribosomal RNA, and transfer RNA. Polypeptides may include amino acid polymers of all lengths and conformations, including antibodies, enzymes, transport proteins, receptor proteins, and structural proteins, among others. Preferred polypeptides are antibodies and enzymes, and particularly monoclonal antibodies. Biological substance also may include synthetic substances that chemically resemble or are derived from biological materials, such as synthetic polypeptides, synthetic nucleic acids (including peptide nucleic acids), and synthetic membranes, vesicles, and liposomes, among others.

Depending on the embodiment, the luminescent metal-ligand complex and/or acceptor may be covalently or noncovalently associated with one or more carrier groups. Covalent association may occur through various mechanisms, including a reactive group, and may involve a spacer for separating the compound from the carrier. Noncovalent association also may occur through various mechanisms, including incorporation of the compound into or onto a matrix, such as a bead or surface, or by nonspecific interactions, such as hydrogen bonding, ionic bonding, or hydrophobic interactions. Noncovalent association also may occur through specific binding pairs, such as avidin and biotin, protein A and immunoglobulins, and lectins and sugars (e.g., concanavalin A and glucose).

“Reactive group” denotes a group capable of forming a covalent attachment with another molecule or substrate. Such other molecules or substrates may include proteins, carbohydrates, nucleic acids, and plastics, among others. Reactive groups vary in their specificity, preferentially reacting with particular functionalities. Thus, reactive compounds generally include reactive groups chosen preferentially to react with functionalities found on the molecule or substrate with which the reactive compound is intended to react. The following reactive groups, among others, may be used in conjunction with the complexes and acceptors described herein:

a) Isothiocyanates, N-hydroxysuccinimide esters, and sulfonylchlorides, which form stable covalent bonds with amines, including amines in proteins and amine-modified nucleic acids

b) Iodoacetamides and maleimides, which form covalent bonds with thiol-functions, as in proteins

c) Carboxyl functions and various derivatives, including N-hydroxybenztriazole esters, thioesters, p-nitrophenyl esters, alkyl, alkenyl, alkynyl, and aromatic esters, and acyl imidazoles

d) Alkylhalides, including iodoacetamides and chloroacetamides

e) Hydroxyl groups, which can be converted into esters, ethers, and aldehydes

f) Aldehydes and ketones and various derivatives, including hydrazones, oximes, and semicarbozones

g) Isocyanates, which react with amines

h) Activated C═C double-bond-containing groups, which can react in a Diels-Alder reaction to form stable ring systems under mild conditions

i) Thiol groups, which can form disulfide bonds and react with alkylhalides (iodoacetamide)

j) Alkenes, which can undergo a Michael addition with thiols, e.g., maleimide reactions with thiols

k) Phosphoamidites, which can be used for direct labeling of nucleosides, nucleotides, and oligonucleotides, including primers on a solid support

5. Example

The invention includes chemical moieties having the following formula, where at least one ligand is a chromophoric bypyridine-based ligand:

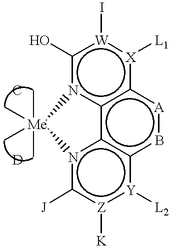

Here, Me may be Ru(II), Os(II), Re(I), Ir(III), or Cr(III), among others. C and D may be chromophoric ligands, although this is not necessary. C and D also may be a bidentate phosphine, arsine-type ligand, such as diphenylphosphinoethane or diphenylphosphinoethylene. Each of C or D may be replaced by two monodentate ligands, such as CO, NHR, or CN, among others. The chromophoric ligand may be a standard bipyridine or phenanthroline (where A and B are CH═CH) ring. The chromophoric ligand also may be a condensed version of these standard rings, where J, K, L₁ and H, I, L₂ are a heterocyclic, aromatic, or aliphatic ring system. The chromophoric ligand also may be a heteroanalog of these standard rings, where one or more of the ring carbons, such as W, X, Y, Z, are replaced by nitrogen, oxygen, sulfur, or another heteroatom. Finally, L₁ and L₂ may be reactive functional groups, which may be separated from the chromophore by a spacer group. The spacer group may be one or more of the following, among others: a hydrocarbon chain, such as —(CH₂)ₙ—, where n=1 to 18, a polyamino acid chain, a peptide chain, or a nucleotide chain. The functional groups may be one or more of the following, among others: isothiocyanate, isocyanate, monochlortriazine, dichlortriazine, aziridine, sulfonylhalogenides, N-hydroxysuccinimide esters, imido-ester, glyoxal and aldehyde for amine- and hydroxyl-functions, as well as maleimides and iodacetamides for thiol-functions. The chemical moiety may be luminescent. The chemical moiety also may be excited with light having a wavelength of about 488 nanometers and/or have an intrinsic polarization greater than about 0.27 (corresponding to a fundamental anisotropy greater than about 0.2).

### B. Assays

Aspects of the invention include assays employing compositions, conjugates, and/or mixtures involving metal-ligand complexes and/or complementary energy transfer acceptors. These assays may include photoluminescence-based assays, such as fluorescence polarization (FP), fluorescence resonance energy transfer (FRET), fluorescence intensity (FLINT), fluorescence lifetime (FLT), total internal reflection (TIR) fluorescence, fluorescence correlation spectroscopy (FCS), and fluorescence recovery after photobleaching (FRAP), among others, as well as analogs based on phosphorescence and/or higher-order transitions.

Luminescence is the emission of light from excited electronic states of luminescent atoms or molecules. Luminescence generally refers to all kinds of light emission, except incandescence, and may include photoluminescence, chemiluminescence, and electrochemiluminescence, among others. In photoluminescence, the excited electronic state is created by the absorption of electromagnetic radiation. In particular, the absorption of radiation excites an electron from a low-energy ground state into a higher-energy excited state. The energy associated with the excited state subsequently is lost through one or more of several mechanisms, including production of a photon through fluorescence, phosphorescence, or other mechanisms. In this application, without limitation, photoluminescence may be used interchangeably with luminescence and fluorescence, and luminophore may be used interchangeably with fluorophore and phosphor. In each case, these terms are intended primarily to designate photoluminescence from compositions, conjugates, and/or mixtures provided by the invention.

Photoluminescence may be characterized by a number of parameters, including extinction coefficient, excitation and emission spectrum, Stokes' shift, luminescence lifetime, and quantum yield. An extinction coefficient is a wavelength-dependent measure of the absorbing power of a luminophore. An excitation spectrum is the dependence of emission intensity upon the excitation wavelength, measured at a single constant emission wavelength. An emission spectrum is the wavelength distribution of the emission, measured after excitation with a single constant excitation wavelength. A Stokes' shift is the difference in wavelengths between the maximum of the emission spectrum and the maximum of the absorption spectrum. A luminescence lifetime is the average time that a luminophore spends in the excited state prior to returning to the ground state. A quantum yield is the ratio of the number of photons emitted to the number of photons absorbed by a luminophore.

Photoluminescence assays (including those listed above) generally involve monitoring aspects (e.g., intensity, polarization, spectrum) of light emitted from a composition and correlating the aspects with properties of an analyte. These aspects may reflect the extinction coefficient, luminescence lifetime, quantum yield, polarization, and/or number of the luminophores in the composition, among others. These quantities, in turn, may reflect the environment and effective geometry of the luminophore, including the proximity and efficacy of quenchers and energy transfer partners, and the size and rotational correlation time of the luminophore. Thus, photoluminescence assays may be used to study, among others, reactions that involve changes in effective size, such as is binding or digestion.

The remainder of this section is divided into two sections that describe applications of the invention to (A) polarization assays and (B) energy transfer assays. These assays may be used for any purpose for which such assays are suited, including drug research, accelerated drug discovery, high-throughput screening, combinatorial chemistry, life science research, genomics, DNA sequencing, and genetic screening, among others. These assays also may be used with apparatus, methods, and compositions described in the above-identified patent applications, which are incorporated herein by reference. For example, luminescence may be detected using high-sensitivity luminescence apparatus, including those described in U.S. patent application Ser. No. 09/062,472, filed Apr. 17, 1998, U.S. patent application Ser. No. 09/160,533, filed Sep. 24, 1998, and PCT Patent Application Ser. No. PCT/US98/23095, filed Oct. 30, 1998. Luminescence also may be detected using high-sensitivity luminescence methods, including those described in PCT Application Ser. No. PCT/US99/01656, filed Jan. 25, 1999, and PCT Patent Application Ser. No. PCT/US99/03678, filed Feb. 19, 1999. Luminescence also may be detected using sample holders optimized for performance with the above-identified high-sensitivity luminescence apparatus and methods, including those described in PCT Patent Application Ser. No. PCT/US99/08410, filed Apr. 16, 1999.

A. Description of Polarization Assays

1. Overview

Luminescence polarization assays involve monitoring the intensity of polarized light emitted from a composition. (Polarization describes the direction of light's electric field, which generally is perpendicular to the direction of light's propagation.) Luminescence polarization assays may be homogeneous and ratiometric, making them relatively insensitive to sample-to-sample variations in concentration, volume, and meniscus shape.

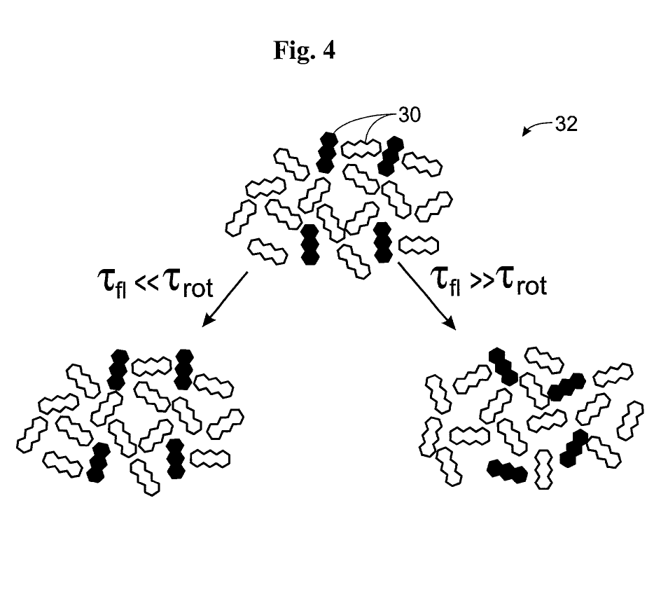

Luminescence polarization assays typically are used to study molecular rotation. FIG. 4 shows how luminescence polarization is affected by molecular rotation. In a luminescence polarization assay, specific molecules 30 within a composition 32 are labeled with one or more luminophores. The composition then is illuminated with polarized excitation light, which preferentially excites luminophores having absorption dipoles aligned parallel to the polarization of the excitation light. These molecules subsequently decay by preferentially emitting light polarized parallel to their emission dipoles. The extent of polarization of the total emitted light depends on the extent of molecular reorientation during the time interval between luminescence excitation and emission, which is termed the luminescence lifetime, a. In turn, the extent of molecular reorientation depends on the luminescence lifetime and the size, shape, and environment of the reorienting molecule. Thus, luminescence polarization assays can be used to quantify hybridization/binding reactions and enzymatic activity, among other applications. In particular, molecules commonly rotate via diffusion with a rotational correlation time τᵣₒₜ that is proportional to their size. Thus, during their luminescence lifetime, relatively large molecules will not reorient significantly, so that their total luminescence will be relatively polarized. In contrast, during the same time interval, relatively small molecules will reorient significantly, so that their total luminescence will be relatively unpolarized.

The relationship between polarization and intensity is expressed by the following equation:

$$P = \frac{{I}_{∥} - {I}_{⊥}}{{I}_{∥} + {I}_{⊥}} \qquad ( 1 )$$

Here, P is the polarization, I_{∥} is the intensity of luminescence polarized parallel to the polarization of the excitation light, and I_{⊥} is the intensity of luminescence polarized perpendicular to the polarization of the excitation light. P generally varies from zero to one-half for randomly oriented molecules (and zero to one for aligned molecules). If there is little rotation between excitation and emission, I_{∥} will be relatively large, I_{⊥} will be relatively small, and P will be close to one-half. (P may be less than one-half even if there is no rotation; for example, P will be less than one if the absorption and emission dipoles are not parallel.) In contrast, if there is significant rotation between absorption and emission, I_{∥} will be comparable to I_{⊥}, and P will be close to zero. Polarization often is reported in milli-P (mP) units (1000×P), which for randomly oriented molecules will range between 0 and 500, because P will range between zero and one-half.

Polarization also may be described using other equivalent quantities, such as anisotropy. The relationship between anisotropy and intensity is expressed by the following equation:

$$P = \frac{{I}_{∥} - {I}_{⊥}}{{I}_{∥} + 2 {I}_{⊥}} \qquad ( 2 )$$

Here, r is the anisotropy. Polarization and anisotropy include the same information, although anisotropy may be more simply expressed for systems containing more than one luminophore. In the description and claims that follow, these terms may be used interchangeably, and a generic reference to one should be understood to imply a generic reference to the other. Generally, polarization has predominated over anisotropy in drug discovery and screening, largely for historical reasons.

The relationship between polarization, luminescence lifetime, and rotational correlation time may be expressed by the Perrin equation:

$$( \frac{1}{P} - \frac{1}{3} ) = ( \frac{1}{{P}_{0}} - \frac{1}{3} ) \cdot ( 1 + \frac{τ}{{τ}_{rot}} ) \qquad ( 3 )$$

Here, P₀ is the polarization in the absence of molecular motion (intrinsic polarization), τ is the luminescence lifetime (inverse decay rate) as described above, and τᵣₒₜ is the rotational correlation time (inverse rotational rate) as described above.

The Perrin equation shows that luminescence polarization assays are most sensitive when the luminescence lifetime and the rotational correlation time are similar. Rotational correlation time is proportional to molecular weight, increasing by about 1 nanosecond for each 2,400 Dalton increase in molecular weight (for a spherical molecule). For shorter lifetime luminophores, such as fluorescein, which has a luminescence lifetime of roughly 4 nanoseconds, luminescence polarization assays are most sensitive for molecular weights less than about 40,000 Daltons. For longer lifetime probes, such as Ru(bpy)₂dcbpy (ruthenium 2,2′-dibipyridyl 4,4′-dicarboxyl-2,2′-bipyridine), which has a lifetime of roughly 400 nanoseconds, luminescence polarization assays are most sensitive for molecular weights between about 70,000 Daltons and 4,000,000 Daltons.

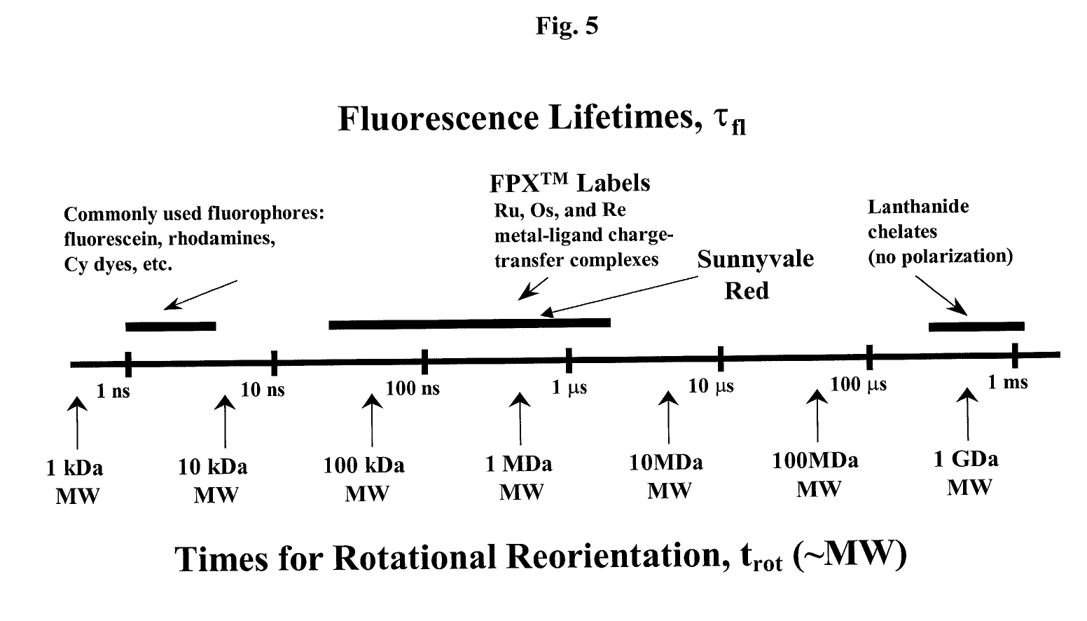

FIG. 5 is a diagram showing the relationship between luminescence lifetime and measurable rotational correlation times in polarization assays, including rotational correlation times rendered measurable in polarization assays provided by the invention.

2. Applications

Aspects of the invention include the use of selected long-lifetime metal-ligand complexes in luminescence polarization assays. Here, as elsewhere in the application, polarization assays encompass any assays involving detection of polarized light, including steady-steady and time-resolved assays, and computation of polarization and anisotropy functions, among others. Preferred complexes possess a high intrinsic polarity and a long luminescence lifetime sufficient for measuring the rotational correlation times of relatively large carriers. Here, the invention was used without limitation to monitor interactions between human serum albumin (HSA) and goat-anti-HSA (IgG) antibodies. The HSA was labeled with a ruthenium-ligand complex polarization probe, Sunnyvale Red™ (SVR™), as described elsewhere in this application. SVR is a preferred polarization probe because it combines long-wavelength absorption and emission and a 200-nm Stokes' shift with a high intrinsic polarity and a long luminescence lifetime (τ=360 ns). The excitation maximum of this dye is 487 nm, so that SVR is ideally suited for excitation with an argon ion laser. The intrinsic polarization P₀ is 0.36 for free SVR, and 0.38 for HSA-labeled SVR (corresponding to fundamental anisotropies of 0.27 and 0.29, respectively). These values were determined by measuring the excitation polarization spectra in vitrified solution (glycerol: water=6:4; −55° C.).

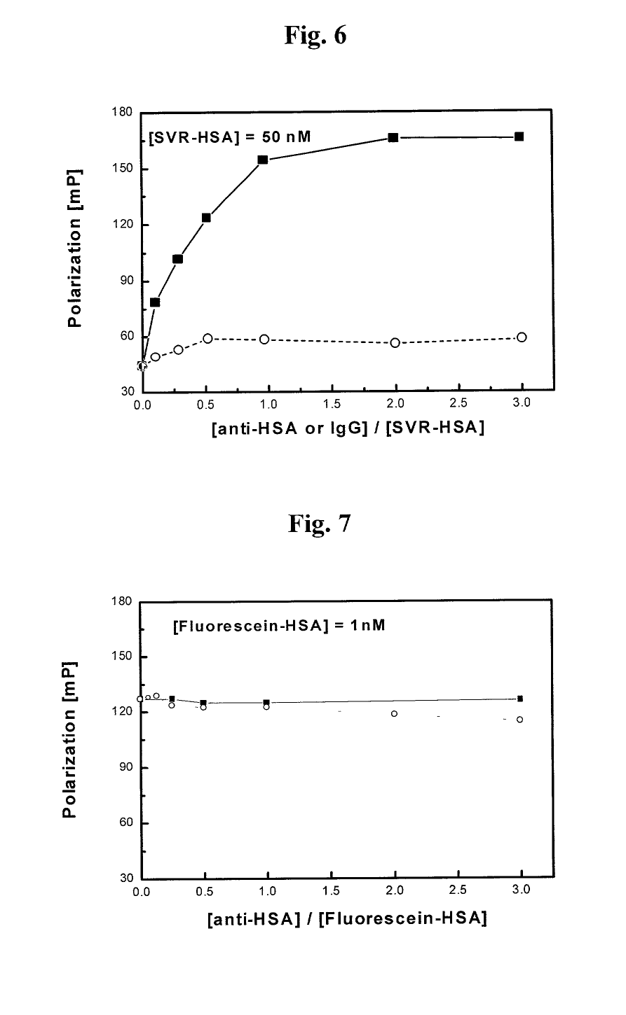

FIG. 6 shows experimental data for this system. Here, changes in the steady-state polarization of SVR⁻IHSA in the presence of various amounts of polyclonal antibody were measured using an Analyst™ light detection platform (LJL BioSystems, Inc.). Specifically, 30 nM of SVR-HSA were premixed with increasing amounts of anti-HSA to yield molar ratios of antigen: antibody of 1:0, 1:0.25, 1:0.5, 1:0.75, 1:1, 1:2, and 1:3. For background correction, an analogous set of samples was prepared using unlabeled HSA. For a nonspecific control, an analogous set of samples was prepared using SVR-HSA and nonspecific anti-HSA antibodies. The various mixtures were incubated for 30 minutes at room temperature. After incubation, the mixtures were transferred in triplicates to a 96-well microplate (Coming Costar), along with PBS (10 mM) and a 10 nM fluorescein reference. Polarizations were measured using the Analyst™ light detection platform. The polarization in mP was calculated for each sample using the formula:

$$P = 1000 P \frac{{I}_{∥} - G \cdot {I}_{⊥}}{{I}_{∥} + G \cdot {I}_{⊥}} \qquad ( 4 )$$

Here, G is a “G factor” that corrects for various instrument artifacts. The G factor is calculated from a known polarization of a standard fluorophore (e.g., fluorescein).

FIG. 6 shows steady-state polarizations for the titration of the SVR-labeled HSA with specific and nonspecific antibody. The polarization increases by more than 100 microplate for the SVR™-labeled HSA, ranging from about 50 mP for the HSA in the absence of antibody to about 165 mP for the saturated immune complex. In contrast, the polarization does not increase significantly for the control sample using nonspecific antibody.

FIG. 7 shows for comparison steady-state polarizations for the titration of fluorescein-HSA under otherwise identical conditions. The polarization is uniformly high for both specific and nonspecific antibody, so that the assay is unable to distinguish differences in the identity or concentration of antibody. This is a reflection of fluorescein's very short luminescence lifetime, which is about 4 ns for free fluorescein and about 2.7 ns for fluorescein covalently attached to protein (fluorescein: HSA D:P ratio=3:1, τ=2.7 ns). Thus, due to this short lifetime, the fluorescein conjugates emit light before the protein carrier rotates significantly, leading to polarized emission, even in the absence of antibody. Generally, fluorescein conjugates are useful in polarization assays only with very small analytes having molecular weights of no more than several kDa. Moreover, if the carrier (protein) includes more than one label, the small Stokes' shift of fluorescein-type labels will cause reabsorption of the emitted photons, further lowering the polarization of the fluorescent protein-conjugate. Thus, the polarization of the fluorescein-HSA-conjugate is reduced to about 130 mP, which is not even close to the theoretical value of about 500 mP. The addition of antibody does not have any effect on the polarization of the fluorescein-HSA.

2. Energy Transfer Assays

A. Overview

Energy transfer is the transfer of luminescence energy from a donor luminophore to an acceptor without emission by the donor. In energy transfer assays, a donor luminophore is excited from a ground state into an excited state by absorption of a photon. If the donor luminophore is sufficiently close to an acceptor, excited-state energy may be transferred from the donor to the acceptor, causing donor luminescence to decrease and acceptor luminescence to increase (if the acceptor is luminescent). The efficiency of this transfer is very sensitive to the separation R between donor and acceptor, decaying as 1/R⁻⁶. Energy transfer assays use energy transfer to monitor the proximity of donor and acceptor, which in turn may be used to monitor the presence or activity of an analyte, among others.

Energy transfer assays may focus on an increase in energy transfer as donor and acceptor are brought into proximity. These assays may be used to monitor binding, as between two molecules X and Y to form a complex X: Y. Here, colon (:) represents a noncovalent interaction. In these assays, one molecule is labeled with a donor D, and the other molecule is labeled with an acceptor A, such that the interaction between X and Y is not altered appreciably. Independently, D and A may be covalently attached to X and Y, or covalently attached to binding partners of X and Y.

Energy transfer assays also may focus on a decrease in energy transfer as donor and acceptor are separated. These assays may be used to monitor cleavage, as by hydrolytic digestion of doubly labeled substrates (peptides, nucleic acids). In one application, two portions of a polypeptide are labeled with D and A, so that cleavage of the polypeptide by a protease such as an endopeptidase will separate D and A and thereby reduce energy transfer. In another application, two portions of a nucleic acid are labeled with D and A, so that cleave by a nuclease such as a restriction enzyme will separate D and A and thereby reduce energy transfer.

Energy transfer between D and A may be monitored in various ways. For example, energy transfer may be monitored by observing an energy-transfer induced decrease in the emission intensity of D and increase in the emission intensity of A (if A is a luminophore). Energy transfer also may be monitored by observing an energy-transfer induced decrease in the lifetime of D and increase in the apparent lifetime of A.

In preferred energy transfer assays provided by the invention, a long-lifetime luminescent metal-ligand complex is used as a donor, and a short-lifetime luminophore or a nonluminophore is used as an acceptor. Suitable metal-ligand-complex donors include those described herein, especially those including ruthenium, osmium, and rhenium. Suitable acceptors also include those described herein, particularly acceptors having absorption spectra rendering them capable of accepting energy transfer from a metal-ligand-complex donor.

Energy transfer may be measured through its effects on the intensity, luminescence lifetime, and/or polarization of donor and/or acceptor emission. These parameters may provide valuable information on the structure, conformation, and proximity of the donor and acceptor in biological assays. Measurement of energy transfer using lifetime is particularly advantageous because lifetime is an intensive quantity and because metal-ligand-complex donor lifetime may be distinguished from background lifetime more easily than may metal-ligand-complex donor intensity be distinguished from background intensity.

Energy transfer may be measured using time-gated and frequency-domain assays, among others. In time-gated assays, the donor is excited using a flash of light having a wavelength near the excitation maximum of D. Next, there is a brief wait, so that electronic transients and/or short-lifetime background luminescence can decay. Finally, donor and/or acceptor luminescence intensity is detected and integrated. In frequency-domain assays, the donor is excited using time-modulated light, and the phase and/or modulation of the donor and/or acceptor emission is monitored relative to the phase and/or modulation of the excitation light. In both assays, donor luminescence is reduced if there is energy transfer, and acceptor luminescence is observed only if there is energy transfer.

2. Applications

Aspects of the invention include the use of selected long-lifetime metal-ligand complexes and complementary acceptors in luminescence energy transfer assays. Here, the invention was used without limitation to monitor interactions between goat-anti-HSA (IgG) antibodies and human serum albumin (HSA). The anti-HSA antibody was labeled with a ruthenium-ligand-complex donor, [Ru(bpy)₂(phen-ITC)]²⁺ (Fair Oaks Red™). The antigen, HSA, was labeled with a nonluminescent absorber, Light Green Yellowish (LGY). Suitable procedures for labeling carriers with donor and acceptor are described in the next section.

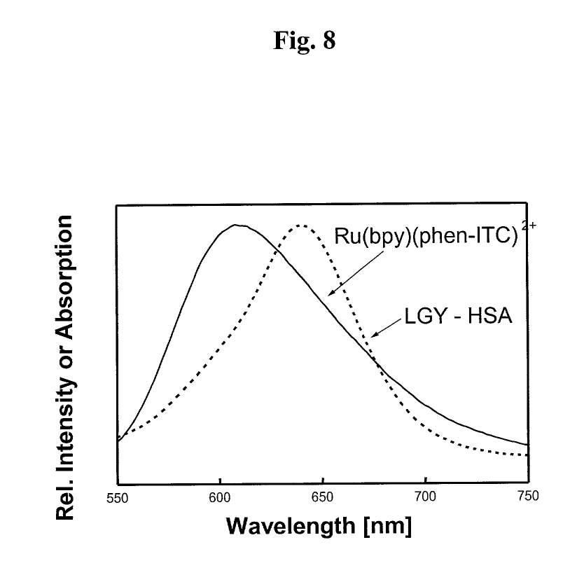

FIG. 8 is a graph showing the emission spectrum of the donor and the absorption spectrum of the acceptor. The figure shows that the metal-ligand complex and LGY form an acceptable donor/acceptor pair because there is sufficient overlap between donor emission and the long-wavelength acceptor absorption.

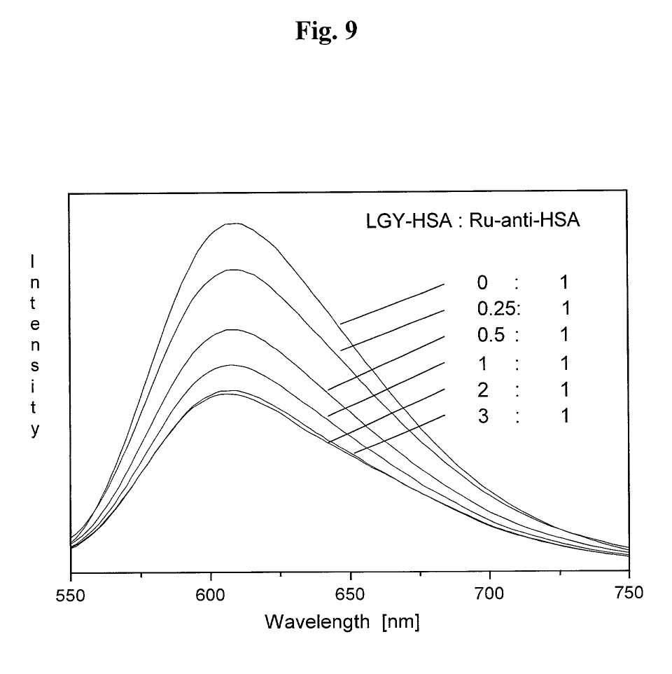

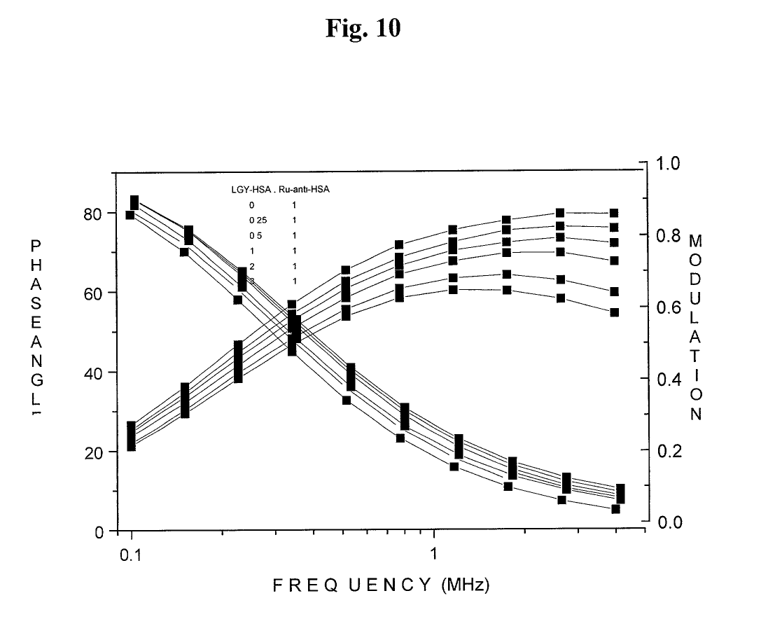

FIGS. 9 and 10 show experimental data for this system. Here, 500 nM Ru-anti-HSA-antibody was premixed with increasing amounts of LGY-HSA to yield molar ratios of 1:0, 1:0.25, 1:0.5, 1:1, 1:2, and 1:3. The mixtures were incubated for 30 minutes at room temperature. After incubation, 200 μL of each mixture were transferred to a 96 well microplate (Coming Costar), along with PBS and a 10 nM fluorescein reference. Energy-transfer induced changes in intensity, phase angle, and modulation were measured using an Analyst™ light detection platform.

FIG. 9 shows relative intensities for the titration of the ruthenium-labeled antibody (donor) with the LGY-labeled human serum albumin (acceptor). Significantly, relative donor intensity decreases as acceptor concentration increases, in response to increased resonance energy transfer.

FIG. 10 shows phase and modulation frequency responses for the titration of the ruthenium-labeled antibody (donor) with the LGY-labeled human serum albumin (acceptor). Significantly, the frequency response curves shift as acceptor concentration increases, in response to increased resonance energy transfer and concomitantly decreased luminescence lifetime.

The phase and modulation method employed here uses sinusoidally modulated light for excitation of the donor. The intensity of the resulting luminescence emission is modulated at the same frequency as the excitation light. However, the emission will lag the excitation by a phase angle (phase) φ, and the intensity of the emission will be demodulated relative to the intensity of the excitation by a demodulation factor (modulation) M. Specifically, the modulation M is the ratio of the AC amplitude to the DC offset for the emission, relative to the ratio of the AC amplitude to the DC offset for the excitation. For a single luminophore, the phase and modulation are related to the luminescence lifetime τ by the following equations:

ωτ+tan(φ) (5)

$$ω τ = \sqrt{\frac{1}{{M}^{2}} - 1} \qquad ( 6 )$$

Here ω is the angular modulation frequency, which equals 27 times the modulation frequency. Thus, the phase and modulation may be used to calculate the luminescence lifetime. (In most measurements, the lifetime of the sample is of no direct interest; in such cases, the phase shift or modulation itself may be used as the measuring parameter.) For maximum sensitivity, the angular modulation frequency should be roughly the inverse of the luminescence lifetime.

It also is possible to perform time-domain measurements using metal-ligand complexes, although such measurements may require a fast light source such as a fast light-emitting diode (LED) or pulsed laser.

### C. Synthetic/Labeling Procedures

Aspects of the invention include materials and procedures for preparing compositions, conjugates, and/or mixtures involving luminescent metal-ligand complexes and/or complementary energy transfer acceptors. These materials and procedures are described below, as follows: (1) reagents and other materials, (2) synthetic procedures for metal-ligand complexes, (3) encapsulation procedures for metal-ligand complexes, (4) >) synthetic procedures for reactive acceptors, and (5) labeling procedures.

1. Reagents and Other Materials

HSA and nonspecific human IgG were obtained from Sigma Chemical Company. Polyclonal IgG specific for HSA (goat anti-HSA) was obtained from O.E.M. Concepts. All other starting reagents were obtained from Aldrich Chemical Company. Sunnyvale Red™ (SVR™) and Fair Oaks Red™ (FOR™)are trademarks of LJL BioSystems, Inc.

2. Synthetic Procedures for Metal-Ligand Complexes

Aspects of the invention include the synthesis of metal-ligand complexes, particularly for use as polarization probes and energy transfer donors. This section describes without limitation the synthesis of representative complexes.

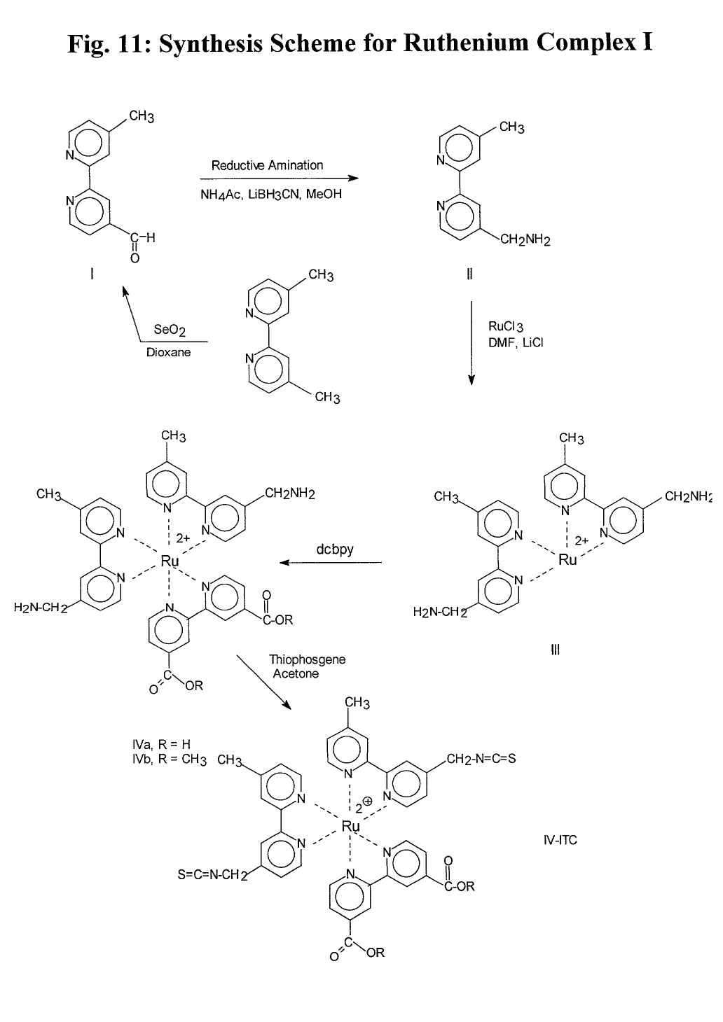

A. Synthesis of Ru(4-aminomethyl-4′-methyl-2,2′-bipyridine)2 (dmcbpy) (Probe 1) (See FIG. 11)

I) Preparation of 4-formyl-4′-methyl-2,2′-bipyridine (I)

4,4′-dimethyl-2,2′-bipyridine (8.0 g, 43 mmol) and selenium dioxide (5.29 g, 48 mmol) were suspended in 440 mL of 1,4-dioxane. The mixture was heated to reflux for 24 hours, until thin-layer chromatography (TLC) showed the termination of the reaction. The hot reaction mixture was filtered, cooled, and filtered again. Dioxane was removed under vacuum, and the resulting solid was dissolved in 730 mL of ethyl acetate at 40° C. The resulting light brown solid was filtered, and the filtrate was washed twice with 140 mL of 1 M sodium carbonate. The organic layer was extracted 3 times with 70 ml of sodium metabisulfite. The aqueous extract was adjusted to pH 10 by adding solid sodium carbonate and extracted four times with 140 mL of methylene chloride. The organic layers were combined, dried over sodium sulfate, and evaporated to dryness. The obtained white solid was dried over P₂O₅ under vacuum. Yield: 46-53%.

II) Preparation of 4-aminomethyl-4′-methyl-2,2′-bipyridine (II)

4-formyl-4′-methyl-2,2′-bipyridine (I) (1.5 g, 7.6 mmol) and ammonium acetate (5.86 g, 76 mmol) were dissolved in 25 mL of methanol, followed by the addition of sodium borohydrocyanide (350 mg, 5.32 mmol). The mixture was stirred at room temperature for 3 days, and a red brown solution was obtained. The reaction was quenched by addition of concentrated hydrochloric acid to adjust the pH to 1. The acidic solution was stirred at room temperature for 1 hour. The resulting light brown solid was filtered. After the methanol was evaporated, a viscous greenish brown residue was obtained. This residue was dissolved in 20 mL of water and extracted 3 times with 25 mL of methylene chloride. The aqueous layer was adjusted to pH 8-9 and extracted 6 times with 25 mL of methylene chloride. The combined organic phases were dried over sodium sulfate, and the solvent was evaporated. 1 mL of 37% hydrochloric acid and 3 mL of methanol were added to the brown oily residue. After the solvent was evaporated, a yellow solid was obtained that was recrystallized in a mixture of isopropanol, cyclohexane, and ethyl acetate. The very hygroscopic product was dried over P₂O₅. Yield: 431 mg, 29%. ¹H NMR in D₂O: 8.648-8.899 (m 2H), 8.295-8.359 (d 2H), 7.742-7.930 (m 2H), 2.687(S 3H), 4.624 (S 2H).

III) Preparation of Ru-bis(4-aminomethyl-4′-methyl-2 2′-bipyridine) (III)

Ruthenium trichloride (41.5 mg, 0.20 mmol) and lithium chloride (56.5 mg, 1.33 mmol) were dissolved in 10 mL of N,N-dimethylformamide (DMF), and 4-aminomethyl-4′-methyl-2,2′-bipyridine (II) (100 mg, 0.42 mmol) was added. The reaction mixture was refluxed for 18 hours. 60 mL of water and 800 mg of sodium carbonate were then added to adjust the pH to 9. The precipitated product was filtered and washed with water until it was almost colorless. A dark solid was obtained. Yield: 71 mg, 62%.

IV) Preparation of Ru-bis(4-aminomethyl-4′-methyl-2,2′-bipyridine)? (dcbpy) (IVa)

Ru-bis(4-aminomethyl-4′methyl) (III) (50 mg) and 4,4′-dicarboxyl-2,2′-bipyridine (dcbpy) (50 mg) were dissolved in 6 mL of ethylene glycol. The mixture was refluxed for 6 hours. After reaction, most of ethylene glycol was removed by heating under argon flow, and 30 mL of water were added, followed by 250 mg of ammonium hexafluorophosphate. A brown product precipitated, which was purified by LH-20 chromatography. Yield: 51 mg, 68%.

V) Preparation of Ru(4-aminomethyl-4′-methyl-2,2′-bipyridine), (dmcbpy) (IVb)

50 mg Ru-bis(4-aminomethyl-4′-methyl-2,2′-bypyridine)-2,2′-bipyridine (III) and 45 mg dmcbpy were suspended in 6 mL of ethylene glycol. The reaction mixture was refluxed for 6 hours. After reaction, most of the ethylene glycol was removed under argon flow, and 20 mL of water were added, followed by 2.5 g of ammonium hexafluorophosphate. A brown product precipitated, which was purified after filtration by LH-20 chromatography. Yield: 25.6 mg, 43%.

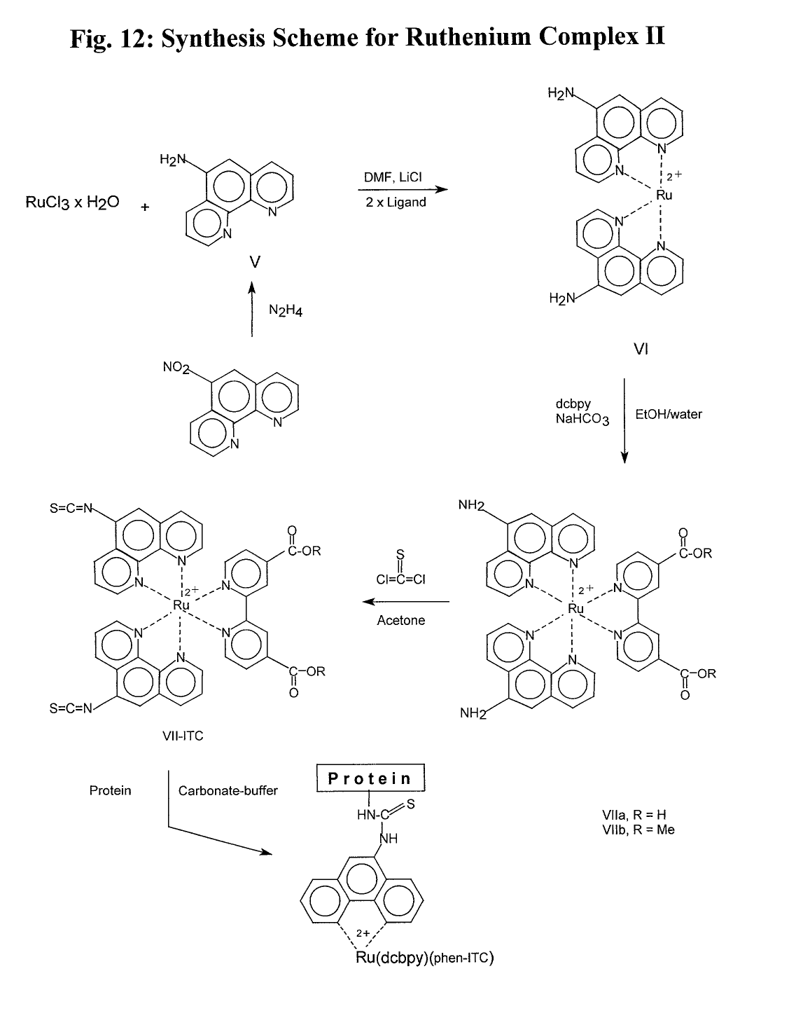

B. Synthesis of tris-Ru(Phen-NH₂)₂ (dmcbpy) (Probe 2) (See FIG. 12)

I) Preparation of aminophenanthroline (Phen-NH₂) (V)

2.0 g of 5-nitro-1,10-phenanthroline and 400 mg of 5% palladium over active carbon were dissolved and suspended in 40 mL of ethanol. 2 mL of hydrazine hydrate dissolved in 40 mL of ethanol were slowly added. The reaction mixture was then heated to 70° C. for 7 hours. Palladium on active carbon was filtered, and the solution was concentrated to about 10 mL. 25 mL of water were added, whereupon a light yellow product precipitated. After filtration, the product was dried overnight under high vacuum. Yield: 1.35g, 81%.

II) Preparation of Ru-bis(aminophenanthroline) (VI)

100 mg of amino-phenanthroline and 53.2 mg of ruthenium trichloride were dissolved in 6 mL of DMF, and 72.4 mg of lithium chloride were added. The reaction was stirred at room temperature for 1.5 hours and heated to 60° C. for 3 hours under an argon blanket. A black solid precipitated. 30 mL of water were added to the mixture. The product was filtered, washed with water, acetone, and methanol, and dried under high vacuum. Yield: 68 mg, 47%.

III) Preparation of Ru-bis(Phen-NH₂)₂(dcbpy) (VIIa)

50 mg of Ru-bis(Phen-NH₂)₂ (VI) were dissolved in 6 mL of ethylene glycol. 40 mg of dcbpy were added, and the solution was refluxed for 3 hours under an argon blanket. After reaction, most of ethylene glycol was evaporated via heating and argon flow until about 1 mL was left. 30 mL of water were added, followed by 600 mg of ammonium hexafluorophosphate. A brown product precipitated. After filtration, the product was purified by LH-20 chromatography using acetone as eluent. The solvent was removed, and the sample was dried under high vacuum for 3 hours. Yield: 82 mg, 90%.

IV) Preparation of tris-Ru(Phen-NH₂)₂(dmcbpy) (VIIb)

50 mg of Ru-bis(Phen-NH₂)₂ (VI) were dissolved in 6 mL ethylene glycol. 40 mg of dmcbpy were added, and the solution was refluxed for 3 hours under an argon blanket. After reaction, most of the ethylene glycol was evaporated via heating and argon flow until about 1 mL was left. 30 mL of water were added, followed by 400 mg of ammonium hexafluorophosphate. A brown product precipitated. After filtration, the product was purified by LH-20 chromatography using acetone as eluent. The solvent was removed, and the sample was dried under high vacuum for 3 hours. Yield: 66 mg, 70%.

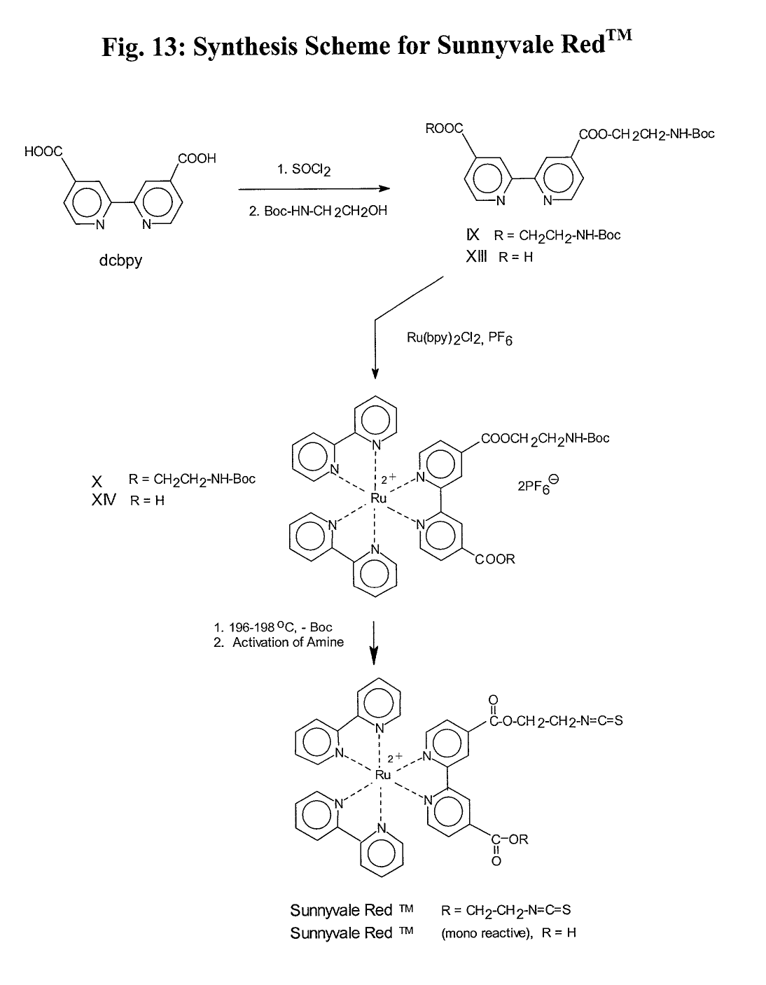

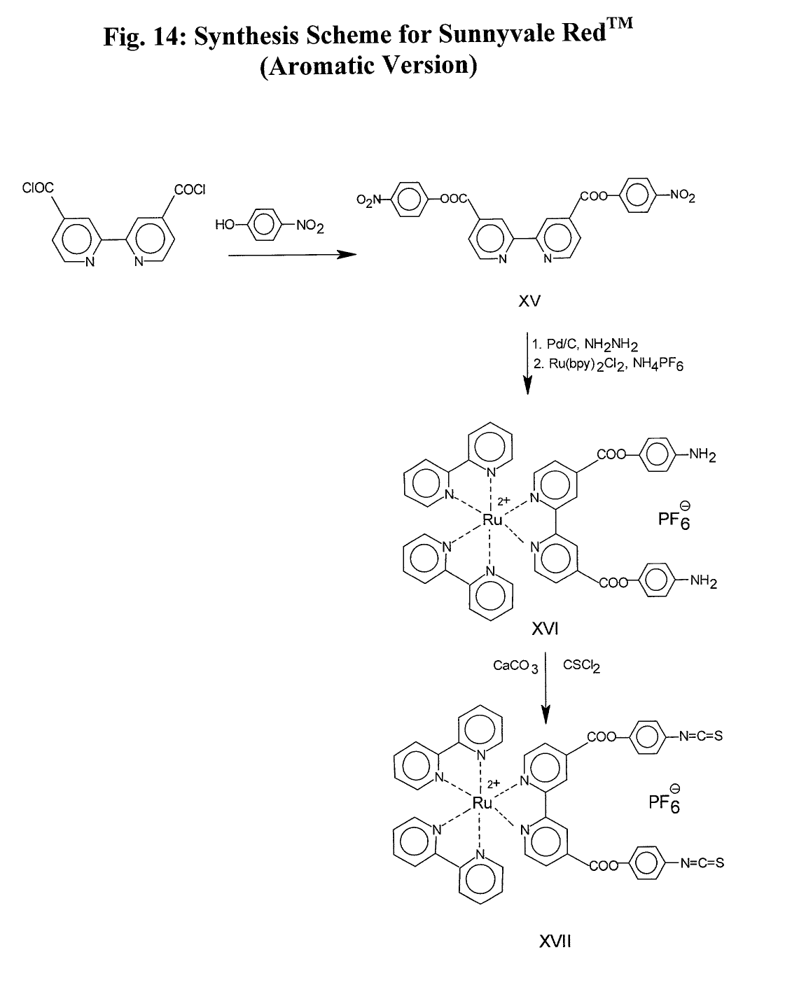

C. Synthesis of Sunnyvale Red-ITC™ (See FIGS. 13-14)

I) Bis-functional-aliphatic-isothiocyanate Version (See FIG. 13)

a) Preparation of 4,4′-dichlorocarbonyl-2,2′-bipyridine (dcbpy-Cl) (VIII)

1.4 g of 4,4′-dicarboxy-2,2′-bipyridine (dcbpy) were suspended in 15 mL of thionyl chloride in a round-bottomed flask equipped with a condenser and a drying tube. The reaction mixture was refluxed for 19 hours until the solution appeared to be clear. Excess thionyl chloride was distilled off, and a light yellow solid product was obtained. The product was dried under high vacuum for 2 hours and used directly for the next reaction step. Yield: 1.56g, 97%.

b) Preparation of 4,4′-[2,2′(di-t-butyloxycarbonylamino)ethoxy carbonyl)]-2,2′-bipyridine (dacbpy-Boc) (IX)

2.5 g of tert-butyl-N-(2-hydroxyethyl)-carbamate were dissolved in 10 mL of dry DMF, and 2 mL of triethyl amine were added. The mixture was slowly added to a solution of 1.56 g of dcbpy-Cl in 10 mL of dry DMF. The reaction mixture was stirred overnight at room temperature. 20 mL of methanol were then added. A light yellow solid product was obtained and filtered. The product was washed twice with water, twice with methanol, and dried under high vacuum for 2 hours. Yield: 2.2 g, 75%. ¹H NMR: 8.90-8.99 (m 4H), 7.94-8.00 (d 2H), 4.43-4.47(t 4H), 3.53-3.58 (m 4H), 1.40 (s 18H).

c) Preparation of Ru(bpy)₂(dacbpy ) (X)

200 mg of Ru(bpy)₂Cl₂ and 330 mg of dacbpy-Boc were suspended in 15 mL ethylene glycol. The reaction mixture was heated to 70-80° C. for 23 hours and then refluxed for 25 minutes under argon. After the reaction, most of the ethylene glycol was evaporated by heating under an argon flow. Heating was continued until 4-5 mL of the solvent were left in the reaction flask. 40 mL of water were added, followed by 4 g of ammonium hexafluorophosphate. The precipitated brown product was filtered and washed with water. The product was purified by neutral alumina column chromatography, using acetone as eluent. Yield: 163 mg, 38%.

d) Preparation of Ru(bpy)₂(dacbpy)-ITC (Sunnyvale Red-ITC) (XII)

46.7 mg of Ru(bpy)₂(dacbpy) were dissolved in 0.6 mL of anhydrous DMF. 31 mg thiocarbonyldiimidazole were added. The solution was stirred at room temperature for 4 hours. SVR-ITC was obtained after LH-20 column chromatography purification, using anhydrous acetone as eluent. Yield: 33.2 mg, 66%.

II) Mono-reactive Version of Sunnyvale Red™ (See FIG. 8)

a) Preparation of 4-carboxy-4′-[2-(t-butyloxycarbonylamino) ethoxycarbonyl)]-2,2′-bipyridine (macbpy-Boc) (XIII)

0.15 mL (1.07 mmol) of Et₃N was added to a solution of 0.2 g (0.71 mmol) dcbpy acid dichloride in 30 mL toluene under argon. The mixture was stirred for 15 minutes at room temperature and heated to 60° C. Then 0.115 g (0.71 mmol) of t-butyloxycarbonylethanolamine in 20 mL toluene was added for 40 minutes at 60° C. under argon. The mixture was heated at this temperature for 15 hours. The hot mixture was filtered; the precipitate was isolated, dried, and afterwards washed with 10 mL water and then again dried. The toluene filtrate was concentrated to 5 mL, and the precipitate was isolated, washed with 5 mL benzene, dried, washed with 10 mL water, and then dried. Yield: 70 mg, 26%. For purification, the product was dissolved in 200 mL ethanol and filtered. The solvent was concentrated to 10 mL, and the precipitate was isolated. Fp: 350-360° C. Found N: 10.8%, calculated N 10.8%. A small amount of di-substituted product (co-product) also was isolated.

b) Preparation of Ru(bpy)₂-4-carboxy-4′[2-(t-butyloxycarbonylamino) ethoxycarbonyl)]-2,2′-bipyridine [PF₆]₂, [Ru(bpy)₂(macdcbpy)][PF₆]₂ (XIV)

47 mg (0.09 mmol) of Ru(bpy)₂Cl₂.2H₂O were suspended in 10 mL of ethanol at room temperature. The mixture was purged with argon, 35 mg (0.091 mmol) of the ligand XIII were added, and the mixture was refluxed for 7 hours under argon atmosphere. After cooling, 5 mL of water and 0.29 g (1.8 mmol) of NH₄PF₆ in 5 mL water were added. The mixture was cooled in the refrigerator overnight, and the red crystalline product was filtered and washed with water and ether. Yield: 40 mg (41%). Found N: 9. 1%, calculated N: 9.0%. λₘₐₓ(abs): 290, 458 nm (EtOH).

III) Sunnyvale Red™ (Aromatic Version) (See FIG. 14)

a) Preparation of 4,4′-p-nitrophenoxycarbonyl-2,2′-bipyridine (npdcbpy) (XV)

350 mg of 2,2′-dicarboxy-4,4′-bipyridine (dcbpy) were dissolved/suspended in 4 mL of thionyl chloride. The mixture was refluxed for 18 hours, until it formed a light yellow homogeneous solution. Thionyl chloride was evaporated and product was dried under high vacuum for an hour. The acid chloride of was stirred in 2 mL anhydrous DMF for 10 minutes. Then 250 mg p-nitrophenol and 0.6 mL anhydrous diusopropyl-ethylamine in 2 mL anhydrous DMF were added slowly. The mixture was slightly heated for 4.5 hours. After cooling and filtration, the product was washed 4 times with methanol and dried under high vacuum overnight. Yield: 408 mg, 59%.

b) Preparation of Ru-bis(2,2′-bipyridine)(4,4′-di-p-aminophenoxycarbonyl-2,2′-bipvridine)[PF₆][Ru(bpy)₂(apdcbpy)] (XVI)

150 mg of npdcbpy (XV) were suspended in 4 mL ethanol. 28.6 mg of 5% palladium on active carbon (catalyst) were added. Then 150 μl hydrazine in 3 mL ethanol were added slowly. The mixture was refluxed for 23 hours until all the starting material was used up. The product was filtered with the catalyst and dried under high vacuum overnight (121.4 mg). 50 mg of this solid product and 40 mg of Ru(bpy)₂Cl₂ were dissolved/suspended in 4 mL of ethylene glycol. The mixture was refluxed for 30 minutes. Most of ethylene glycol was evaporated by heating under an argon stream. Afterwards 15 mL of water were added and the catalyst was filtered. Then 1.5 g ammonium hexafluorophosphate was added. A reddish brown product precipitated and after filtration the product was purified by neutral alumina column chromatography using acetone as eluent. The first reddish brown band was collected. Yield: 96.9 mg, ˜80%.

c) Preparation of [Ru(bpy)₂(apdcbpy)]-ITC (XVII)—Aromatic Version of Sunnyvale Red™

26.3 mg of Ru(bpy)₂(apdcbpy) was dissolved in 0.8 mL anhydrous acetone. 15.2 mg of calcium carbonate was added. The mixture was stirred at room temperature for 10 minutes. Then 30 μl of thiophosgene was added. The solution was stirred at room temperature for 2 hours and refluxed for an hour. After filtration, solvent was evaporated and product was dried under high vacuum for 2 hours. Yield: 28.3 mg, 100%.

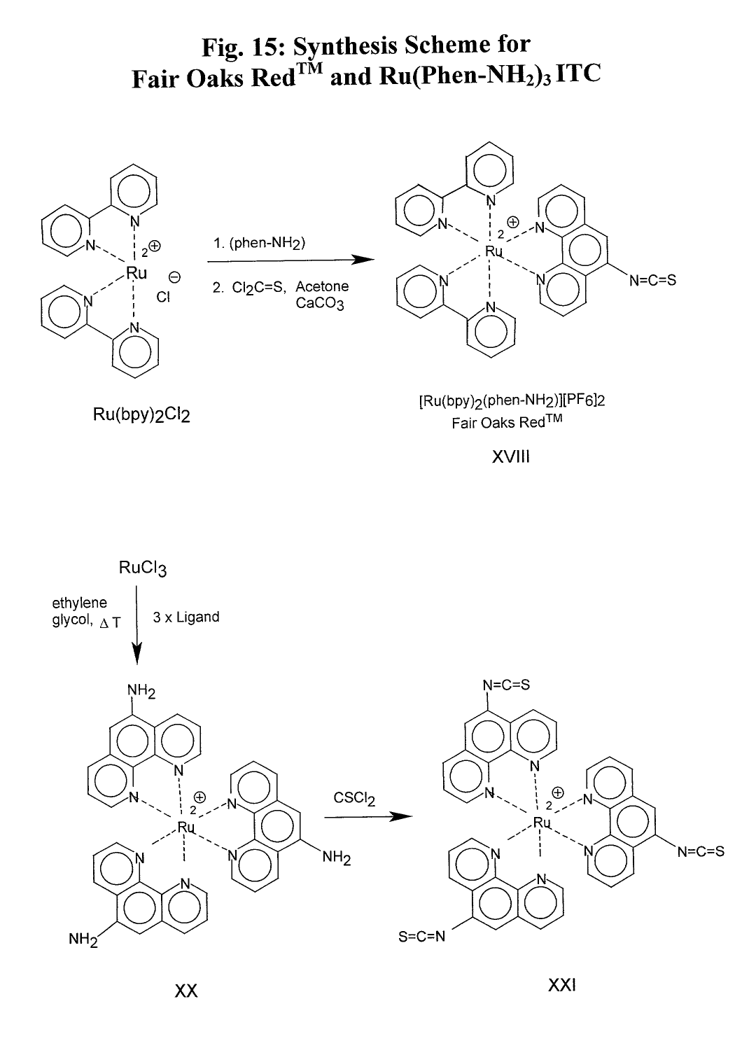

D. Synthesis of Fair Oaks Red™ (See FIG. 15)

I) Preparation of Ru(bpy)₂(Phen-NH₂)PF₆ (Fair Oaks Red™) (XVIII)

50 mg of Ru(bpy)₂Cl₂ and 21 mg of Phen-NH₂ (see b)) were dissolved in 2 mL of water and 2 mL of methanol. The mixture was refluxed for 8 hours under argon. After the methanol was evaporated, 40 mg of ammonium hexaflourophosphate were added. A red brown solid precipitated and was dried under high vacuum overnight. Yield: 84 mg, 90%.

II) Preparation of [Ru(bpy)₂(Phen-NH₂)-ITC][PF₆]₂ (XIX)

20 mg of Ru(bpy)₂(Phen-NH₂)PF₆ were dissolved in 0.5 mL of anhydrous acetone. 12 mg of calcium carbonate was added. The solution was stirred at room temperature for 15 minutes. Then 5 μl of thiophosgene was added. The reaction mixture was stirred at room temperature for an hour and heated to reflux for 2.5 hours under argon blanket. After filtration the solvent was evaporated. The product was dried under high vacuum for 2 hours. Yield: 20.2 mg, 97%.

E. Synthesis of [Ru(Phen-NH₂)₃][PF₆]₂-ITC (See FIG. 15)

I) Preparation of [Ru(Phen-NH₂)₃][PF₆]₂ (XX)

70 mg of ruthenium trichloride, 210 mg of Phen-NH₂ and 95.3 mg of lithium chloride were dissolved in 12 mL of ethylene glycol. The mixture was refluxed for 2 hours under argon. Most of ethylene glycol was evaporated via heating under an argon stream until 2-3 mL remained. 40 mL of water were added followed by 300 mg of ammonium hexafluorophosphate. A dark brown solid precipitated which was purified by neutral alumina column chromatography using acetonitrile/toluene (5:1) as eluent. The first reddish brown band was collected. 195 mg product was obtained. Yield: 60%.

II) Preparation of [Ru(Phen-NH₂)₃-ITC[PF₆]₂ (XXI)

20 mg of Ru(Phen-NH₂)₃ PF₆ were dissolved in 1 mL anhydrous acetone. 21 mg of calcium carbonate were added. The mixture was stirred at room temperature for 15 min. Then 15 μl of thiophosgene were added. The solution was stirred at room temperature for an hour and heated to reflux for 2.5 hours under argon. After filtration and evaporation of the solvent, the product was dried under high vacuum for 2 hours. Yield: 21 mg, 92%.

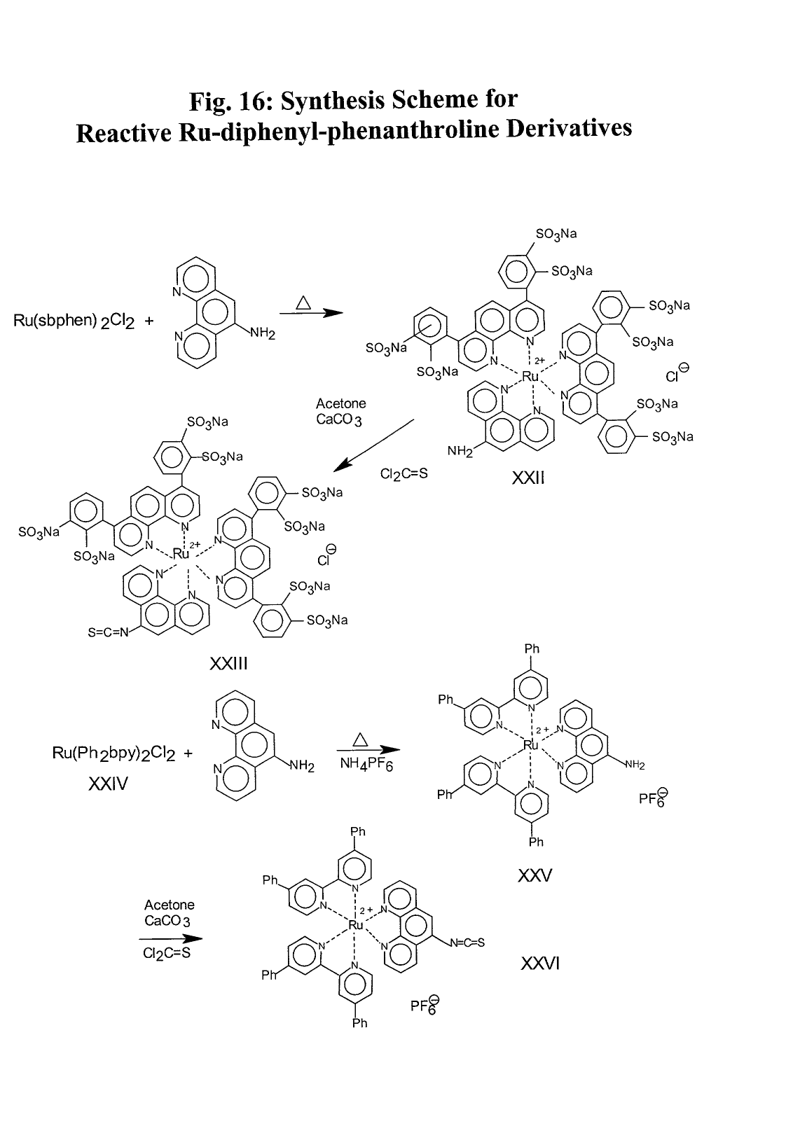

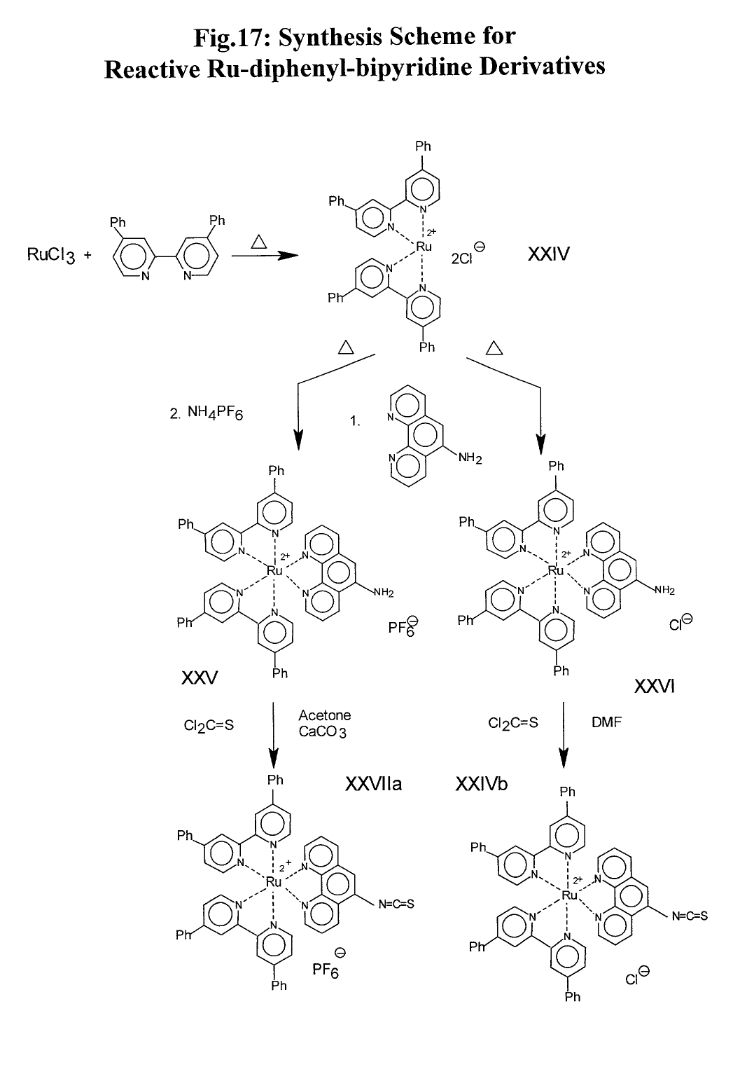

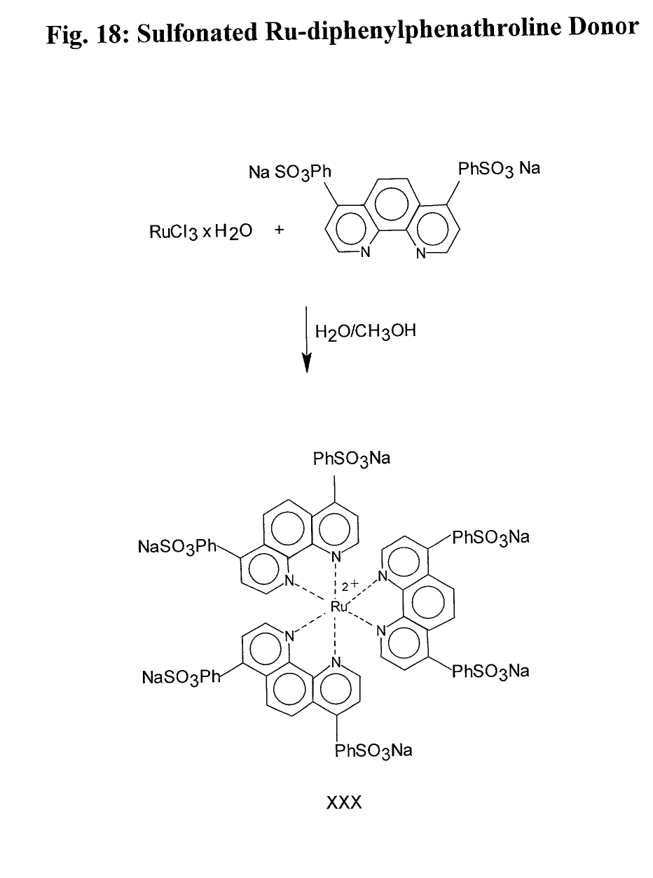

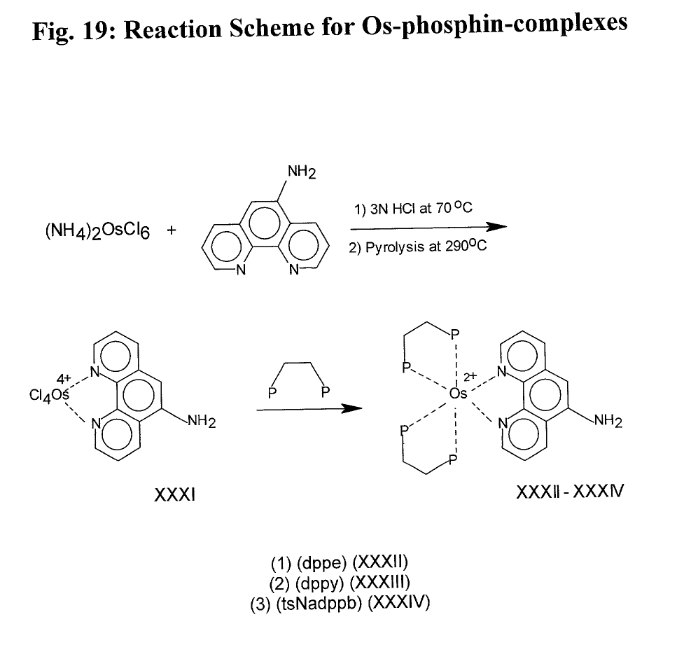

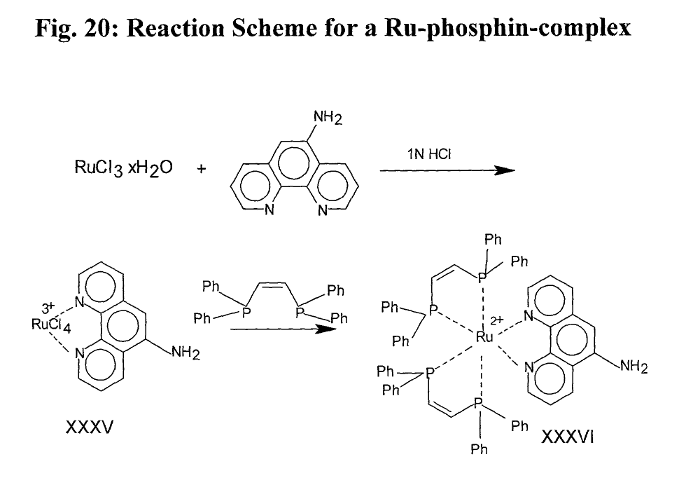

F. Synthesis of [Ru(sbphen)₂(Phen-NH₂)-ITC][PF₆]₂ (See FIG. 16)

I) Preparation of Ru-bis(4,7-disulfodiphenylphenanthroline)(5-amino-phenanthroline)Cl₂, [Ru(sbphen)₂(Phen-NH₂)Cl₂ (XXII)

50 mg of Ru(sbphen)₂Cl₂ and 16.6 mg of 5-aminophenanthroline were dissolved in 50 mL water and 20 mL methanol. The mixture was refluxed for 18 hours under argon atmosphere. The purple solid that remained in solution was filtered and solvent was evaporated. The product was recrystallized from water and ethanol and dried under vacuum for 3 hours. Yield: 36.3 mg, 63%.

II) Preparation of [Ru(sbphen)₂(Phen-NH₂)-ITC][PF₆]₂ (XXII)

10 mg of Ru(sbphen)₂(Phen-NH₂)Cl₂ was dissolved in 0.5 mL of anhydrous DMF and 5 mg calcium carbonate were added. The solution was stirred at room temperature for 20 minutes and then 5 μl of thiophosgene were added. After that the mixture was stirred at room temperature for another hour and then slightly heated for 2 hours. After filtration, the solvent was removed under reduced pressure. Yield: 100%.

G. Synthesis of [Ru(Ph₂bpy)₂(Phen-NH₂)]-ITC (Probe 8) (See FIG. 16)

I) Preparation of Ru-bis(4,4′-diphenyl-2,2′-bipyridine)Cl₂, [Ru(Ph₂bpy)₂]Cl₂ (XXIV)

300 mg of 4,4′-diphenyl-2,2′-bipyridine and 110 mg of ruthenium trichloride were dissolved in 5 mL DMF. The mixture was heated to 70° C. for 1 hour and then refluxed for 2 hours. Afterwards most of the DMF was removed under reduced pressure until about 1 mL remained. 5 mL acetone and 10 mL of water were added and the precipitate was filtered and washed with water until colorless. Yield: 278 mg, 73%.

II) Preparation of [Ru(Ph₂bpy)₂(Phen-NH₂)][PF₆]₂ (XXV)

60 mg of Ru(Ph₂bpy)₂Cl₂ and 20 mg of 5-amino-phenanthroline were dissolved/suspended in 4 mL of DMF. The reaction mixture was refluxed for 18 hours. Afterwards most of the DMF was evaporated and 8 mL water were added followed by 700 mg of ammonium hexafluorophosphate. The reddish brown product, which precipitated was filtered and purified by LH-20 column chromatography using acetone as eluent. The first reddish brown band was collected. Yield: 87.9 mg, 96%.

III) Preparation of [Ru(Ph₂bpy)₂(Phen-NH₂)][Cl]₂ (XXVI)

80 mg Ru(Ph₂bpy)₂Cl₂ and 26 mg of 5-aminophenanthroline were dissolved in 4.5 mL DMF. The mixture was refluxed for 16 hours. Most of the solvent was removed to about 1 mL. The precipitating product was purified by LH-20 column chromatography using methanol as eluent. The first reddish brown band was collected. Yield: 87 mg, 87.5%.

IV) Activation of [Ru(Ph₂bpy)₂(Phen-NH₂)][PF₆]₂ (XXVIIa)

200 mg of [Ru(Ph₂bpy)₂(Phen-NH₂)][PF₆]₂ were dissolved in 3 mL anhydrous acetone. 200 mg of calcium carbonate were added, and the mixture was stirred at room temperature for 10 min. Afterwards, 80 μl of thiophosgene were added, and the solution was stirred at room temperature for 1.5 hours and subsequently refluxed for 1.5 hours. After filtration, the solvent was evaporated, and the product was isolated. Yield: 100%.

V) Activation of [Ru(Ph₂bpy(Phen-NH₂)]Cl₂ (XXXVIIb)

15 mg of Ru(Ph₂bpy)₂(Phen-NH₂)Cl₂ was dissolved in 0.4 mL anhydrous DMF. 10 mg sodium carbonate was added. The mixture was stirred in an ice bath for 10 minutes. Then 10 μl thiophosgene was slowly added. The solution was first stirred in an ice bath for 15 minutes and than at room temperature for 1.5 hours. After filtration excess thiophosgene was removed under reduced pressure. Yield: 100%.

H. Synthesis of [Ru(Ph₂bpy)₂(dmcbpy)][BPh₄]₂ (XVIII) (Probe 9)

147 mg of Ru(Ph₂bpy)₂Cl₂ and 50 mg dmcbpy were dissolved/suspended in 3 mL of ethylene glycol. The mixture was refluxed for 30 minutes under an argon atmosphere. Most of the ethylene glycol was evaporated while heating under the argon stream. 6 mL water and 2 mL methanol were added to homogenize the solution and after that 170 mg of sodium tetraphenylborate were added. After cooling to 4° C. for 3 hours the product was filtered and washed twice with water and dried under high vacuum overnight. Yield: 285 mg, 94%).

I. Synthesis of [Ru(bpy)₂(dmcbpy)][PF₆] (XIX) (Probe 10)

190 mg of Ru(bpy)₂Cl₂ and 110 mg of dmcbpy were dissolved/suspended in 3 mL ethylene glycol. The mixture was heated to reflux for 30 minutes under argon atmosphere. Most of ethylene glycol was evaporated by heating in an argon stream. The product was purified by column chromatography on neutral alumina using methanol as eluent. After evaporating of methanol the product was dissolved in 10 mL of water and 3 g of ammonium hexafluorophosphate were added. The reddish brown product precipitate was isolated, washed twice with water and dried under high vacuum for 3 hours. Yield: 319 mg, 83.4%.

J. Synthesis of Tris-(4,7-disulfodiphenylphenanthroline)RuCl₂, Ru(sbphen)₃Cl₂ (XXX) (Probe 11)

40.9 mg of RuCl₃xH₂O and 363.9 mg (3.5 equivalent) of bathophenanthroline disulfonic acid, disodium salt hydrate were dissolved in 15 mL of water. The solution was refluxed for 4.5 days. After filtration and evaporation of the solvent, the product was purified by column chromatography using LH-20 and water as eluent. The dark reddish brown band was collected and the majority of the solvent was evaporated under reduced pressure and a dry product was obtained after lyophilization overnight. Yield: 94.8 mg, 23%.

K. General Synthesis of Os(phen-NH₂)diphosphin Complexes

I) Synthesis of Os(phen-NH₂)Cl₄ (XXXI)

250 mg of ammonium hexachloroosmate were dissolved in 12.5 mL of 3N HCl, and the solution was warmed at 70° C. 113.2 mg of 5-aminophenanthroline were dissolved in 3 mL of 3N HCl, and the resulting solution was added slowly into the osmate solution. The reaction mixture was kept at 70° C. for another 10 minutes after addition. A brown product precipitated. After cooling at 0° C. for 3 hours, the product was filtered and washed 3 times with 3N HCl, once with water, and 4 times with acetone. The product was dried under high vacuum for 7 hours. Yield: 292.7 mg.

The product was placed in a round-bottomed flask, which was then positioned in the center of a salt bath formed from a mixture of sodium nitrate and potassium nitrate melted at 290° C. A white fume formed at the beginning, and pyrolysis lasted for 17 hours. The product turned from reddish brown to black. After pyrolysis, the product was stirred in 20 mL of 3N HCl at room temperature for 3 hours. After filtration, the procedure was repeated in 20 mL of acetone for 2 hours, and the product was filtered and dried under high vacuum for 1 hour. Yield: 223.1 mg.

II) Synthesis of Os(Phen-NH₂)(diphosphine)₂ PF₆ (XXXII-XXXIV)

20 mg of Os(Phen-NH₂)Cl₄ and 3-3.5 equivalent of diphosphine ligand were dissolved/suspended in 5 mL of ethylene glycol. The mixture was refluxed for 16 hours under an argon blanket. After reaction, most of the ethylene glycol was removed by heating under argon flow until about 1 mL was left. 20 mL of water were then added, followed by 400 mg of ammonium hexafluorophosphate. After filtration, the product was first purified by LH-20 column chromatography using acetone as eluent and then by neutral alumina column chromatography using acetonitrile/toluene (5:1) as eluent. The first band of product (yellowish fluorescent) was collected.

L. Synthesis of Ru(Phen-NH₂)(dppy)₂ PF₆

I) Synthesis of Ru(Phen-NH₂)Cl₄ (XXXV)

200 mg of 5-aminophenanthroline were dissolved in 1.5 mL of 1N HCl, and 218 mg of ruthenium trichloride were added. The flask was sealed and allowed to react at room temperature for 6 days. A black product was obtained, which was washed 3 times with water after filtration and dried under high vacuum for 30 minutes. Yield: 427.9 mg.

II) Synthesis of Ru(Phen-NH₂)(dppy)₂ PF₆ (XXXVI)

50 mg of Ru(Phen-NH₂)Cl₄ and 120 mg of dppy were dissolved/suspended in 5 mL of ethylene glycol. The mixture was refluxed for 26 hours under argon blanket. After reaction, most of the ethylene glycol was evaporated via heating under argon flow until about 1 mL was left. 20 mL of water were then added, followed by 2 g of ammonium hexafluorophosphate. A brown product precipitated out immediately. After filtration, the product was purified by LH-20 column chromatography using acetone as eluent.

3. Encapsulation Procedures for Metal-Ligand Complexes

Aspects of the invention also include the encapsulation of metal-ligand complexes in beads, macromolecules, dendrimers, and/or other carriers, particularly for use as energy transfer donors. This encapsulation can be achieved using generally known procedures, for example, by in-situ incorporation (i.e., during the synthesis of the polymer) or post-synthetic incorporation of the guest molecule. For post-synthetic encapsulation into dendrimers, it may be necessary to remove the outer shell of the dendrimer before encapsulation and then to resynthesize the outer shell after encapsulation.

4. Synthetic Procedures for Reactive Acceptors

Aspects of the invention also include the synthesis of (reactive) acceptor molecules. This section describes without limitation the synthesis of representative acceptors.

A. Synthesis of Light Green SF Yellowish-Sulfonyl Chloride (XXXVII) (See FIG. 3)

500 mg of Light Green SF Yellowish and 1.2 g of phosphorus pentachloride were thoroughly mixed in a round-bottomed flask. The solid mixture was stirred at room temperature for 18 hours, when the mixture turned viscous. The product then was transferred into 30 mL of ice water and extracted five times with 50 mL of chloroform. The combined organic layers were dried using anhydrous sodium sulfate. After filtration and evaporation of the solvent, the product was further dried overnight under high vacuum. Yield: 297 mg, 60%.

B. Synthesis of Naphthol Blue Black

I) Isothiocyanate (XXXVIII)

30 mg of Naphthol Blue Black were dissolved in 1 mL of anhydrous DMF. 10 mg of calcium carbonate were added, and the mixture was stirred at room temperature for 15 minutes. 10 μL of thiophosgene then were added, and the reaction mixture was stirred at room temperature for 3 hours and afterwards at 70° C. for 3 hours. After filtration and removal of excess thiophosgene under reduced pressure, the remaining solid was used for labeling.

II) Sulfonyl-chloride (XXXIX)

200 mg of Naphthol Blue Black and 320 mg of phosphorus pentachloride were thoroughly mixed in a round-bottomed flask. The solid mixture was stirred overnight at room temperature. The activated dye was transferred into ice water and extracted 3 times with 20 mL of chloroform. The combined organic layers were dried over anhydrous sodium sulfate. After filtration and evaporation of the solvent, the product was dried under high vacuum for 40 minutes. Yield: 86 mg, 44%.

C. Synthesis of Fast Green FCF-Sulfonyl Chloride (XL)

600 mg of Fast Green FCF and 1 g of phosphorus pentachloride were thoroughly mixed for 5 minutes in a mortar and afterwards transferred to a round bottom flask. The viscous solid mixture then was stirred at room temperature for 24 hours. The product was transferred into ice water and extracted 5 times with chloroform. The combined organic layers were dried over anhydrous sodium sulfate. After filtration and evaporation of the solvent, the resulting solid was dried under vacuum for 3 hours. Yield: 132.9 mg, 22.3%.

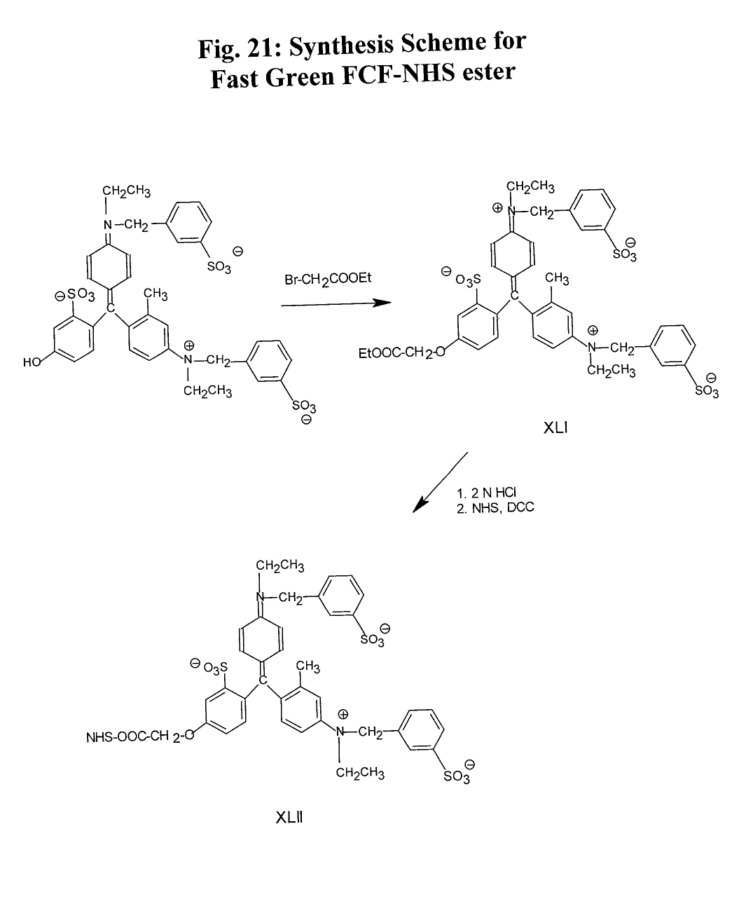

D. Synthesis of O-acetylated-Fast Green FCF-NHS Ester (See FIG. 21)

I) Synthesis of O-methylacetyl-Fast Green FCF (XLI)

200 mg of Fast Green FCF were dissolved in 3 mL of DMF and 0.3 mL of water. 60 mg of potassium carbonate were added, followed by 120 mg of bromomethylacetate. The mixture was stirred at room temperature for 6 hours, and the product was purified by preparative high-pressure liquid chromatography (HPLC) using a gradient of water and acetonitrile. The methylester was cleaved with 2 N HCl, and the carboxyl-containing compound was isolated and purified using preparative TLC.

II) O-acetyl-Fast Green FCF-NHS Ester (XLII)

a) Using NHS and DCC

100 mg of acetylated-Fast Green FCF were dissolved in 1.5 mL of anhydrous DMF. 26 mg of N-hydroxysuccinimide (NHS) were added, followed by 47 mg of 1,3-dicyclohexylcarbodiimide. The mixture was stirred at room temperature, and the reaction was monitored by TLC or analytical HPLC.

b) Using TSU

2.8 mg of acetylated-Fast Green FCF were dissolved in 80 μL of anhydrous DMF. 3.7 mg of O-(N-succinimidyl)-N,N,N′N′-tetramethyluronium tetrafluoroborate (TSU) were added followed by 2 μL of diisopropyl ethyl amine. The mixture was stirred at room temperature for 2 hours. The solution was used directly for labeling a protein.

5. Labeling Procedures

Aspects of the invention also include the labeling of carriers with metal-ligand complexes and/or acceptors. Such carriers may include proteins, antibodies, polymers, and drugs. This section describes without limitation procedures for labeling protein carriers with metal-ligand complexes and acceptors.

A. Labeling of Carriers with Metal-Ligand Complexes for Polarization and Energy Transfer

I) Protocol 1

In a first protocol, human serum albumin (HSA) was labeled with isothiocyanates of selected ruthenium metal-ligand complexes by adding a 30-fold molar excess of the Ru-ITC in 50 mL of DMF to 0.75 mL of a stirred protein solution (0.2 M carbonate buffer, pH 8.9-9.2), followed by a 3-hour incubation at room temperature and purification of the labeled protein by gel filtration chromatography on Sephadex G-25, using 10-50 mM phosphate-buffered saline (PBS, pH 7.2).

II) Protocol 2

In a second protocol, HSA was labeled with Sunnyvale Red-ITC by adding a 30-fold molar excess of the dye in 60 μL of dry DMF to 940 μL of a stirred HSA solution (0.1 M carbonate buffer, pH 8.9). The mixture was incubated for 4 hours at room temperature and purified by gel filtration chromatography on Sephadex G-25, using 10 mM PBS (pH 7.2). The dye:protein ratio of the Sunnyvale Red-HSA conjugate was determined to be 2.3, with a protein concentration of 2.0 mg/mL.

III) Protocol 3

In a third protocol, HSA was labeled with Sunnyvale Red™ by dissolving 0.25 mg of HSA in 60 μL of 100 mM sodium carbonate buffer (pH 8.9). 0.1 mg (1 vial) Sunnyvale Red™ dissolved in 10 μL of anhydrous DMF was added to the stirred protein solution. The reaction mixture was stirred at room temperature for 3 hours. The conjugate was obtained after dialysis in a MWCO 7000 Slide-A-Lyser cassette against 10 mM PBS buffer (pH 7.4) for 16 hours at 4° C.

The dye:protein ratio was determined as follows. The volume of solution was about 330 μL after dialysis. The dye concentration was determined by absorption spectroscopy to be 1.39×10⁻⁵ M, with A₄₇₉=0.78175 and E(SVR) ˜15,000. The HSA concentration was determined by BCA assay (Pierce Reagent) to be 0.75 mg/mL, which was 3.0×10⁻⁶ M. SVR:HSA=4.6:1

IV) Protocol 4

In a fourth protocol, Ru(bpy)₂(phen-ITC)(PF₆)₂ (Fair Oaks Red™ (FOR™)) was conjugated to anti-HSA by adding a 30-molar excess of the dye to 3 mg of anti-HSA in 800 μL of 0.1 M carbonate buffer (pH 8.9), followed by overnight incubation at 4° C. The reaction mixture was dialyzed for 12 hours against 10 mM PBS using a dialysis membrane with a molecular weight cutoff of 12,000-14,000 kDa. The dye:protein ratio of the FOR-anti-HSA conjugate was determined to be 2:1, with a protein concentration of 3.2 mg/mL.

B. Labeling of Carriers with Acceptors

1) Protocol 1

HSA was labeled with LGY-sulfonyl chloride by adding a 30-molar excess of the dye in 60 μL of dry DMF in small aliquots to 940 μL of a stirred solution of HSA in 0.1 M carbonate buffer (pH 8.9), followed by 1.5 hours incubation at room temperature. The conjugate was purified by gel filtration chromatography on Sephadex G-25 with 10 mM PBS (pH 7.2). The dye:protein ratio of the LGY-HSA conjugate was 6, with an estimated protein concentration of 1.4 mg/mL.

II) Protocol 2

2.7 mg of streptavidine (SA) were dissolved in 600 μL of 100 mM sodium carbonate buffer (pH 8.9). 2.8 mg of TSU-activated acetylated-Fast Green FCF were added. The mixture was stirred at room temperature for 4 hours. The conjugate was purified by gel filtration chromatography on Sephadex G-25 with 10 mM PBS (pH 7.2). The dye:protein ratio was 6.7.

Although the invention has been disclosed in its preferred forms, the specific embodiments thereof as disclosed and illustrated herein are not to be considered in a limiting sense, because numerous variations are possible. For example, singular terms used herein do not preclude the use of more than one of the associated element, and embodiments utilizing more than one of a particular element are within the spirit and scope of the invention. Applicants regard the subject matter of their invention to include all novel and nonobvious combinations and subcombinations of the various elements, features, functions, and/or properties disclosed herein. No single feature, function, element or property of the disclosed embodiments is essential. The following claims define certain combinations and subcombinations of features, functions, elements, and/or properties that are regarded as novel and nonobvious. Other combinations and subcombinations may be claimed through amendment of the present claims or presentation of new claims in this or a related application. Such claims, whether they are broader, narrower, equal, or different in scope to the original claims, also are regarded as included within the subject matter of applicants' invention. +C

We claim:

1. A composition of matter comprising a photoluminescent compound having a high intrinsic fundamental polarization and the following structure: 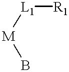
wherein:
M is a long-lifetime luminophore;
L₁ is 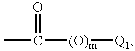
where m is 0 or 1, and Q is alkyl or aryl;
R₁ is selected from the group consisting of —N═C═S and 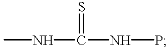
P is selected from the group consisting of proteins, polynucleotides, antibodies, beads, and solid supports; and
B is selected from the group consisting of H, COOH, and 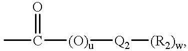
where R₂ is a reactive group or a reactive group coupled to P, u is 0 or 1, w is 0 or 1 and Q₂ is alkyl or aryl.

2. The composition of claim 1, wherein M is a multivalent metal-ligand complex.

3. The composition of claim 1, wherein the composition is bifunctional.

4. The composition of claim 1, wherein each of Q₁ and Q₂ is independently selected from the group consisting of an aromatic ring system and —(CH₂)ᵥ—, where v is 1-10.

5. The composition of claim 1, wherein the lifetime of the compound is at least about 10 nanoseconds.

6. The composition of claim 1, wherein M comprises a metal selected from the group consisting of ruthenium, osmium, and rhenium.

7. The composition of claim 1, wherein M is asymmetric.

8. The composition of claim 1, wherein M comprises a plurality of cyclic moieties, each cyclic moiety selected from the group consisting of phenanthroline, bipyridine, bipyrazine, bipyrimidine, and dipyridophenazine.

9. The composition of claim 1, wherein M comprises Ru coordinated to three bipyridine moieties.

10. The composition of claim 9, wherein L₁ is

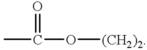

11. The composition of claim 1, wherein each of R₁ and B comprises —N═C═S.

12. The composition of claim 1 further comprising:
an energy transfer acceptor suitable for use with the compound in a fluorescence energy transfer assay.

13. The composition of claim 1, wherein the reactive group is selected from the group consisting of —N═C═S, NHS, and L₂—N═C═S.

14. The composition of claim 1, wherein the quantum yield of the compound is at least about 4 percent.

15. A composition of matter comprising a photoluminescent compound having a high intrinsic fundamental polarization and the following structure: 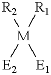
wherein:
M is a long lifetime luminophore;
R₁ is selected from the group consisting of —N═C═S, 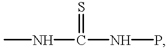
and 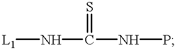 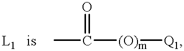
where m is 0 or 1, and Q₁ is alkyl or aryl;
P is selected from the group consisting of proteins, polynucleotides, antibodies, beads, and solid supports;
E₁ is an electron-withdrawing group;
R₂ is selected from the group consisting of H, —N═C═S, L₂—N═C═S 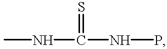
and 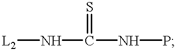 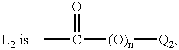
n is 0 or 1, and Q₂ is alkyl or aryl; and
E₂ is selected from the group consisting of H and an electron-withdrawing group.

16. The composition of claim 15, wherein M comprises a metal selected from the group consisting of ruthenium, osmium, and rhenium.

17. The composition of claim 15, wherein R₂ is L₂—N═C═S or

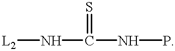

18. The composition of claim 15, wherein E₂ is an electron-withdrawing group.

19. The composition of claim 15, wherein E₁ is an electron-withdrawing group selected from the group consisting of ester, carboxyl, sulfonate, sulfonic ester, quartenary ammonium, and nitro.

20. The composition of claim 15, wherein M is asymmetric.

21. The composition of claim 15, wherein R₁ is —N═C═S.

22. The composition of claim 15, wherein each of Q₁ and Q₂ is independently selected from the group consisting of an aromatic ring system, and —(CH₂)ᵥ—,where v is 1-10.

23. The composition of claim 15, wherein the composition is bifunctional.

24. The composition of claim 15 further comprising;
an energy transfer acceptor suitable for use with the compound in a fluorescence energy transfer assay.

25. The composition of claim 15, wherein the quantum yield of the compound is at least about 4 percent.
